(19) United States
(12) Patent Application Publication
(10) Pub. No.: US 2023/0302196 A1
(43) Pub. Date: Sep. 28, 2023

(54) METHODS OF FABRICATING 3D HIERARCHICAL NANOFIBER SCAFFOLDS WITH STRUCTURAL AND/OR COMPOSITIONAL GRADIENTS

(71) Applicant: Board of Regents of the University of Nebraska, Lincoln, NE (US)

(72) Inventors: Jingwei XIE, Omaha, NE (US); Shixuan CHEN, Elkhorn, NE (US)

(21) Appl. No.: 18/015,094

(22) PCT Filed: Jul. 20, 2021
(86) PCT No.: PCT/US2021/042310

§ 371 Date: Jan. 9, 2023

Related U.S. Application Data
(60) Provisional application No. 63/054,042, filed on Jul. 20, 2020.

(51) Int. Cl. A61L 27/26; A61L 27/56; A61L 27/34; A61L 27/52; A61L 27/54

(52) U.S. Cl. CPC A61L 27/26; A61L 27/34; A61L 27/52; A61L 27/54; A61L 27/56; A61L 2300/418; A61L 2400/12; A61L 2430/02; A61L 2430/06

(57) ABSTRACT

Nanofiber structures are provided as well as methods of use thereof and methods of making.

This application claims priority under 35 U.S.C. §119(e) to U.S. Provisional Pat. Application No. 63/054,042, filed Jul. 20, 2020. The foregoing application is incorporated by reference herein.

This invention was made with government support under Grant No. R01 GM123081 awarded by the National Institutes of Health. The government has certain rights in the invention.

### FIELD OF THE INVENTION

This application relates to the fields of nanofiber structures. More specifically, this invention provides nanofiber structures, methods of synthesizing, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Complex three-dimensional (3D) assembly of nanofibers represents ubiquitous extracellular matrix (ECM) in most human tissues (Stevens, et al. (2005) Science 18:1135-1138). Nanofiber structures and scaffolds have been widely used to mimic the architecture of ECM in native tissues (Wang, et al. (2018) Sci. Adv. 4:eaat4537; MacQueen, et al. (2018) Nat. Biomed. Eng., 2(12):930-941; Carlson, et al. (2016) Nat. Commun., 7:10862; Chen, et al. (2018) Adv. Drug Del. Rev., 132:188-213). However, current methods for producing nanofiber structures and scaffolds are insufficient for mimicking the complexity of ECM. Accordingly, new methods for the fabrication of nanofiber structures are needed.

### SUMMARY OF THE INVENTION

In accordance with the instant invention, nanofiber structures or scaffolds with gradient pore sizes, compositional gradients, and/or varied alignments are provided. Methods of making the nanofiber structures are also provided.

In certain embodiments, nanofiber structures comprising regions with different pore sizes are provided. In certain embodiments, methods of synthesizing a nanofiber structure comprising regions with different pore sizes are provided. In certain embodiments, the method comprises electrospinning a nanofiber structure comprising a first nanofiber region or layer comprising a polymer and a first amount or concentration of a surfactant and a second nanofiber region or layer with a polymer and a second amount or concentration of a surfactant; and expanding the electrospun nanofiber structure by gas foaming, wherein the first amount or concentration of a surfactant is different than the second amount or concentration of a surfactant. In certain embodiments, the nanofiber structure comprises more than two nanofiber regions. In certain embodiments, the surfactant is an amphiphilic block copolymer, particularly a poloxamer such as poloxamer 407. In certain embodiments, the polymer is a hydrophobic polymer such as polycaprolactone. In certain embodiments, the polymer and the surfactant are the same in all of the regions of nanofiber structure.

In certain embodiments, nanofiber structures comprising regions with different nanofiber alignments are provided. In certain embodiments, methods of synthesizing a nanofiber structure comprising regions with different nanofiber alignments are provided. In certain embodiments, the method comprises electrospinning a nanofiber structure comprising a first nanofiber region or layer with a polymer with a first nanofiber alignment and a second nanofiber region or layer with a polymer with a second nanofiber alignment; and expanding the electrospun nanofiber structure by gas foaming, wherein the first nanofiber alignment is different than the second nanofiber alignment. In certain embodiments, the nanofiber structure comprises more than two nanofiber regions. In certain embodiments, the nanofiber polymer is a hydrophobic polymer such as polycaprolactone. In certain embodiments, the nanofiber polymer is the same in all of the regions of nanofiber structure. In certain embodiments, the nanofiber alignments are selected from the group consisting of aligned nanofibers, partially aligned nanofibers, random nanofibers, and entangled nanofibers.

In certain embodiments, nanofiber structures comprising a compositional gradient of at least one agent are provided. In certain embodiments, methods of synthesizing a nanofiber structure comprising a compositional gradient of at least one agent are provided. In certain embodiments, the method comprises electrospinning a nanofiber structure (e.g., mat or membrane) comprising radially aligned nanofibers; expanding the electrospun nanofiber structure by gas foaming; and adding the at least one agent to the center of the expanded nanofiber structure, wherein the at least one agent radially diffuses through the expanded nanofiber structure. In certain embodiments, the nanofiber polymer is a hydrophobic polymer such as polycaprolactone.

In certain embodiments, the nanofiber structures of the instant invention and/or methods of making the nanofiber structures further comprise other modifications. In certain embodiments, the nanofiber structures of the instant invention and/or methods of making the nanofiber structures further comprise coating the nanofibers and/or the nanofiber structure with a hydrogel or gelatin; mineralizing the nanofibers and/or the nanofiber structure; crosslinking or thermally treating the nanofibers and/or nanofiber structure; and/or adding cells, tissues, and/or agents to the nanofiber structure.

In accordance with another aspect of the instant invention, methods of using the nanofiber structures are provided. For example, the nanofiber structures may be used to enhance wound healing, build tissue constructs, promote tissue regeneration (e.g., bone and/or cartilage regeneration), reduce, inhibit, prevent, and/or eliminate infection, local delivery of drugs, and/or inhibit bleeding.

### BRIEF DESCRIPTION OF THE DRAWINGS

FIGS. 1A-1J show the preparation and characterization of a nanofiber scaffold with a gradient in gap or pore distance. FIG. 1A: Schematic illustrating a 1-mm thick PCL nanofiber mat comprising four regions containing 2%, 1%, 0.5% and 0% Pluronic® F-127 from bottom to top (each region: 250 µm). FIG. 1B: Photograph of an expanded PCL nanofiber scaffold comprising four regions containing 2%, 1%, 0.5% and 0% Pluronic® F-127 from bottom to top. FIG. 1C: The length percentage of each region of expanded 3D PCL nanofiber scaffolds. FIG. 1D: The morphology of cross-sections (Y-Z plane) of each region of expanded PCL nanofiber scaffolds. FIG. 1E: The gap distance between two adjacent layers in each region of expanded PCL nanofiber scaffolds. FIG. 1F: The compressive stress-strain curves of each region of expanded PCL nanofiber scaffolds. FIG. 1G: The maximum compressive stress of each region of expanded PCL nanofiber scaffolds. FIG. 1H: Schematic illustrating the expansion process of PCL nanofiber mats with blending different amounts of Pluronic® F-127. The Pluronic® F-127 additive not only enhances the hydrophilicity and water penetration of PCL nanofiber mats, and but also stabilizes the formed H₂ bubbles. The small H₂ bubbles can merge and grow larger in volume with increasing the concentration of Pluronic® F-127, resulting in a higher expansion rate. FIG. 11: Schematic showing the oxygen level of each region in expanded PCL nanofiber scaffolds in cell culture medium. FIG. 1J: The density of each region of the expanded 3D PCL nanofiber scaffold. * p<0.05, **p<0.01.

FIGS. 2A-2G provide the processes of BMSCs seeding and differentiation. FIG. 2A: The expanded nanofiber scaffolds (width 10 mm, length 10 mm, height 6-8 mm) were immersed into 1×10⁷ BMSCs suspension solution, then treated with vacuum for 10 seconds, which was repeated 3 times. FIG. 2B: The BMSCs seeding efficiency of scaffolds treated with or without vacuum for 10 seconds. FIG. 2C: The BMSCs seeded expanded nanofiber scaffolds were placed into 0.1% agar pre-treated 24-well culture plate and continuously cultured for 1, 3, 6 and 9 days. FIG. 2D: Schematic illustrating the definition of porous and dense groups for chondrogenic and osteogenic differentiation. FIG. 2E: Schematic of BMSCs seeded porous and dense scaffolds for chondrogenic differentiation and osteogenic differentiation for 7 and 14 days. FIGS. 2F and 2G: SEM images show the cross-section of porous and dense scaffolds, respectively, used for chondrogenic osteogenic differentiation.

FIGS. 3A-3M show BMSCs cultured on expanded nanofiber scaffolds with dense and porous structures. FIG. 3A: Schematic illustrating BMSCs culture on expanded nanofiber scaffolds with porous structure (left), the cross-section of BMSCs on nanofiber scaffolds with porous structure (right). FIG. 3B: Schematic illustrating BMSCs culture on expanded nanofiber scaffolds with dense structure (left), the cross-section of BMSCs seeded nanofiber scaffold with dense structure (right). FIG. 3C: Schematic illustrating the top layer, medium layer, and central layer of BMSCs on nanofiber scaffolds. FIG. 3D: EF-5 staining of BMSCs on nanofiber scaffolds with porous and dense structures after 7 days of culture in the proliferating medium. FIG. 3E: The quantification of EF-5 positive BMSCs seeded on nanofiber scaffolds with porous and dense structures after 7 days of culture in the proliferating medium. FIG. 3F: The relative expression of HIF-1α of BMSCs on nanofiber scaffolds with porous and dense structures after culture in the chondrogenic differentiation medium for 7 and 14 days. FIGS. 3G and 3H: The relative expression of Cyclin D1, CDK1, Caspase 3, and Bcl-2 on BMSCs on nanofiber scaffolds with dense and porous structures after culture in the chondrogenic differentiation medium for 7 and 14 days, respectively. FIG. 3I: The heatmap visualizes the chondrogenic and osteogenic differentiation related gene expression profiles of BMSCs on nanofiber scaffolds with dense and porous structures. P-day x-y: Sample y of BMSCs on porous scaffolds after x days of differentiation. D-day-x-y: Sample y of BMSCs on dense scaffolds after x days of differentiation. FIGS. 3J and 3L: The relative expression of aggrecan, SOX-9, and Collagen type 2 of BMSCs on nanofiber scaffolds with dense and porous structures after chondrogenic and osteogenic differentiation for 7 and 14 days, respectively. FIGS. 3K and 3M: The relative expression of RUNX-2, ALP, OCN, and OPN of BMSCs on nanofiber scaffolds with dense and porous structures after chondrogenic and osteogenic differentiation for 7 and 14 days, respectively.

FIGS. 4A and 4C show the alizarin Red S staining of differentiated BMSCs after osteogenic and chondrogenic differentiation for 7 and 14 days, respectively. FIGS. 4B and 4D show the safranin O staining (B, D) of differentiated BMSCs after osteogenic and chondrogenic differentiation for 7 and 14 days.

FIG. 5 shows the quantification of differentiated BMSCs in the central, middle, and top layers of porous and dense nanofiber scaffold after 14 days of chondrogenic differentiation.

FIGS. 6A-6G show the fabrication and characterization of expanded nanofiber scaffolds with dual gradations in both gap distances and fiber organizations. FIG. 6A: Schematic illustrating the 1-mm thick PCL fiber mat composed of aligned (bottom), partially aligned (middle), and random (top) nanofibers. FIG. 6B: Photograph of expanded nanofiber scaffolds composed of aligned (bottom), partially aligned (middle), and random (top) nanofibers. FIG. 6C: The morphology of cross-sections (Y-Z plane) of each region of expanded nanofiber scaffolds composed of aligned (bottom), partially aligned (middle), and random (top) nanofibers. Bottom row: the corresponding highly magnified SEM images showing the nanofiber orientation in each region of expanded nanofiber scaffolds composed of aligned (bottom), partially aligned (middle), and random (top) nanofibers of top row. FIG. 6D: The length percentage of each region of expanded nanofiber scaffolds composed of aligned (bottom), partially aligned (middle), and random (top) nanofibers. FIG. 6E: The gap distance between two adjacent nanofiber layers in each region of expanded nanofiber scaffolds composed of aligned (bottom), partially aligned (middle), and random (top) nanofibers. FIG. 6F: Images of nanofibers in each region of expanded nanofiber scaffolds composed of aligned (bottom), partially aligned (middle), and random (top) nanofibers indicate the angle mapping of fiber orientations. FIG. 6G: The angle distribution of nanofiber orientations in each region of expanded nanofiber scaffolds composed of aligned (bottom), partially aligned (middle), and random (top) nanofibers.

FIGS. 7A-7I show the preparation and characterization of expanded nanofiber scaffolds with compositional gradients. FIG. 7A: The fluorescent images show a 3D radially aligned nanofiber scaffold with a gradation in rhodamine 6G content along the radial direction. The rhodamine 6G gradient was formed by dripping 3 µL or 9 µL of 0.3% rhodamine aqueous solution to the center of scaffolds. False-color images of 3D scaffolds consisting of radially aligned nanofibers after dripping 3 µL and 9 µL 0.3% rhodamine aqueous solution to the center of scaffolds (bottom). FIG. 7B: The confocal image indicates dripping was able to load drugs throughout the whole scaffold from top to bottom. FIG. 7C: Quantification of the fluorescence intensity in each region labeled in FIG. 7A. FIG. 7D: Schematic illustrating ten samples were collected from each region by 1-mm punch. FIG. 7E: Quantification of total released rhodamine 6G from ten collected samples of each region labeled in FIG. 7D. FIG. 7F: Rhodamine 6G release profiles from 3D radially aligned nanofiber scaffolds after dripping 3 µL and 9 µL of 0.3% rhodamine 6G aqueous solution to the center. FIG. 7G: Rhodamine 6G release profiles from 3D radially aligned nanofiber scaffolds after dripping 9 µL of 0.3% rhodamine 6G solution containing 0.5% and 1% Gel-MA to the center without and with UV crosslinking. 0.5% Gel-MA: 0.3% rhodamine 6G solution containing 0.5% Gel-MA without UV crosslinking. 0.5% Gel-MA+UV: 0.3% rhodamine 6G solution containing 0.5% Gel-MA with UV crosslinking. 1% Gel-MA+UV: 0.3% rhodamine 6G solution containing 1% Gel-MA with UV crosslinking. FIG. 7H: The fluorescent images show a 3D radially aligned nanofiber scaffold with dual gradations in rhodamine 6G and FITC-BSA contents along the radial direction from the center to the edge. FIG. 7I: Rhodamine 6G and FITC-BSA release profiles from 3D radially aligned nanofiber scaffolds with dual gradations in rhodamine 6G and FITC-BSA contents along the radial direction from the center to the edge.

FIGS. 8A-8F show applications of 3D radially aligned nanofiber scaffolds with a bFGF gradient along the radial direction for wound healing by recruiting cells towards the center. FIG. 8A: Schematic illustrating the 3D scaffold (8 mm) consisting of radially aligned nanofibers with gradations in the bFGF content that enhances wound healing by guiding and accelerating cell migration. The in vitro wound model (8 mm) is created by punching the Gel-MA hydrogel with GFP-labeled human dermal fibroblasts seeded on the surrounding area. FIG. 8B: Fluorescent images show GFP-labeled human dermal fibroblasts around the 8-mm hole. FIG. 8C: Fluorescent images show the migrated GFP-labeled human dermal fibroblasts into 3D scaffolds after 4 and 8 days of culture. FIGS. 8D and 8E: The quantification of cell counts and surface coverage (%) per scaffold after 4 and 8 days of culture, respectively. FIG. 8F: Schematic illustrating the possible migration routes of GFP-labeled human dermal fibroblasts from the surface of the Gel-MA hydrogel to 3D scaffolds. *p<0.05, **p<0.01. RAS: radially aligned nanofiber scaffolds.

FIG. 9A: Photograph shows a 3D scaffold that is inserted into the hole of GFP-labeled fibroblasts seeded Gel-MA hydrogel. FIG. 9B: The cumulative release of bFGF from radially aligned nanofiber scaffolds with a bFGF gradient. FIG. 9C: The timeline shows the time for adding free bFGF, and the total added free bFGF is equivalent to the amount of bFGF released from radially aligned nanofiber scaffolds with a bFGF gradient within 7 days. RAS: radially aligned nanofiber scaffolds.

FIG. 10A: Schematic illustrating a 1-mm thick PCL nanofiber mat that consists of four regions of nanofibers containing 1%, 0.6%, 0.3%, and 0% rhodamine 6G from bottom to top. FIG. 10B: Photographs of bottom view, side view, and top view of a 1-mm thick PCL nanofiber mat composed of four regions of fibers containing 1%, 0.6%, 0.3%, and 0% rhodamine 6G from bottom to top. FIG. 10C: Rhodamine 6G release profiles from each region of 1-mm thick PCL nanofiber mat. FIG. 10D: The 1-mm thick rhodamine 6G-loaded nanofiber mat were expanded through rapid depressurization of subcritical CO₂ fluid.

### DETAILED DESCRIPTION OF THE INVENTION

Herein, nanofiber structures or scaffolds with gradient pore sizes, compositional gradients, and/or varied alignments are provided. Methods of making and methods of using the nanofiber structures are also provided. Nanofiber structures can be used for many purposes including, but not limited to: regenerative medicine, wound healing, and as a delivery mechanism for cells, pharmaceuticals, and other biologics.

The present invention provides compositions and methods for creating 3D nanofiber structures by converting 2D nanofiber structures (e.g., mats or membranes) into 3D hierarchical assemblies with structural and/or compositional gradients. Briefly, elements of the present study include, without limitation: i) gas-foaming to expand 2D nanofiber structures (e.g., 2D electrospun nanofiber mats or membranes) in the third dimension; ii) transforming 2D nanofiber structures into 3D nanofiber structures with hierarchical structure and/or controlled fiber alignment; iii) incorporating a surfactant (e.g., Pluronic® F-127) into nanofiber mats to significantly increase hydrophilicity and subsequent gas bubble stability, thereby resulting in a faster expansion rate of membranes; and/or iv) coating expanded nanofiber scaffolds (e.g. using a gelatin coating) to increase elasticity and compressibility.

By decreasing the amount of surfactant (e.g., Pluronic® F-127) incorporated into 2D nanofiber structures (e.g., mats or membranes) in each successive layer or region, 2D nanofiber structures can be converted into 3D nanofiber structures with a gradient in pore or gap sizes after the gas-foaming expansion process as each successive layer expanded less than the previous layer. For example, as demonstrated herein, PCL/Pluronic® F-127 nanofiber membranes were prepared by sequential deposition of PCL nanofibers with incorporation of 2%, 1%, 0.5% and 0% Pluronic® F-127 from bottom to top on the rotating mandrel. After the gas-foaming expansion, the 3D nanofiber structure possessed a pore or gap size gradient. The length percentage of each region after expansion significantly increased with increasing the amount of blended Pluronic® F-127 from 0 to 2%.

Herein, expanded electrospun nanofiber structures, particularly via a modified gas-foaming technique, and methods of making and use thereof are provided. Expanded nanofiber structures possess significantly higher porosity than traditional two-dimensional nanofiber mats or membranes, while simultaneously maintaining nanotopographic cues. In certain embodiments, the distribution of gap widths and layer thicknesses are directly dependent on the processing time of nanofiber mats or membranes within the gas bubble forming solution. As seen herein, the expanded nanofiber structure of the instant invention possesses superior properties with regard to inhibiting and/or preventing bleeding and/or hemorrhaging.

In accordance with the instant invention, nanofiber structures with gradient pore sizes, varied alignments, and/or composition gradients and methods of making and using the same are provided. For example, the nanofiber structures of the instant invention may comprise gradient pore sizes; varied nanofiber alignments; composition gradients; gradient pore sizes and composition gradients; varied nanofiber alignments and composition gradients; pore sizes and varied nanofiber alignments; or gradient pore sizes, varied nanofiber alignments, and composition gradients. The 3D or expanded nanofiber structures of the instant invention may be referred to herein as nanofiber structures, nanofiber scaffolds, or nanofiber assembly.

The nanofibers of the instant invention can be fabricated by any method. In a particular embodiment, the nanofiber scaffolds comprise electrospun nanofibers. In a particular embodiment, the structure may comprise aligned fibers (e.g., uniaxially aligned), partially aligned, random fibers, and/or entangled fibers. In a particular embodiment, the nanofiber structure comprises random fibers. In a particular embodiment, the nanofiber structure comprises aligned fibers (e.g., uniaxially, radially, vertically, or horizontally).

While the application generally describes nanofibers (fibers having a diameter less than about 1 µm (e.g., average diameter)) and the synthesis of nanofiber scaffolds, the instant invention also encompasses microfibers (fibers having a diameter greater than about 1 µm (e.g., average diameter)) and the synthesis of microfibrous scaffolds. The nanofiber scaffolds may be crosslinked (e.g., chemically crosslinked) and/or thermally treated. In a particular embodiment, the nanofibers and/or nanofiber scaffolds are air plasma treated.

In certain embodiments, the 3D or expanded nanofiber structure of the instant invention comprises regions with different size pores or gaps. In certain embodiments, the nanofiber structure comprises at least a first region and a second region, wherein the pore size (e.g., average pore size (e.g., by volume)) of the first region is different (e.g., larger or smaller) than the pore size (e.g., average pore size (e.g., by volume)) of the second region. In certain embodiments, the pore size of the first region is at least 25%, 50%, 75%, 100%, 150%, 200%, 250%, 300%, 350%, 400%, 450%, 500% or more, larger or smaller (e.g., by volume) than the pore size of the second region. The nanofiber structure may comprise two or more regions. For example, the nanofiber structure may comprise 2, 3, 4, 5, 6, 7, 8, 9, 10, or more regions. The regions of the nanofiber structure can be characterized by their different pore sizes. In other words, each region of the nanofiber structure will have a different (e.g., larger or smaller) pore size (e.g., average pore size) than each adjacent region. In certain embodiments, all of the regions of the nanofiber structure have different pore sizes. The regions of the nanofiber structure may be arranged as layers within the nanofiber structure. The layers or regions within the nanofiber structure may have the same or different thicknesses. The layers or regions within the nanofiber structure may have the same length and width. In certain embodiments, the layers or regions within the nanofiber structure increase or decrease in pore size (e.g., average pore size). In other words, a gradient with regard to pore size is created. For example, in the direction of expansion, the pore size of each region or layer may increase or decrease throughout the nanofiber structure.

In accordance with the instant invention, methods for synthesizing the nanofiber structures of the instant invention comprising different pore or gap sizes are provided. In certain embodiments, the method comprises sequential deposition of different regions during the synthesis process of the 2D nanofiber structure (e.g., mat or membrane). In certain embodiments, the method comprises electrospinning a first nanofiber region (e.g., mat or membrane) with a polymer and a first amount of a surfactant and electrospinning a second nanofiber region (e.g., mat or membrane) with a polymer and a second amount of a surfactant and expanding the electrospun structure to create the nanofiber structures of the instant invention comprising different pore sizes, wherein the first amount of surfactant is different than the second amount of surfactant. In certain embodiments, the electrospun structure is expanded using a gas foaming technique. In certain embodiments, the method comprises electrospinning two or more regions, e.g., 2, 3, 4, 5, 6, 7, 8, 9, 10, or more regions.

The polymer may be the same or different through the different regions. In a particular embodiment, the polymer is the same through the regions of the nanofiber structure. In certain embodiments, the polymer is a hydrophobic polymer. In certain embodiments, the polymer comprises polycaprolactone (PCL).

The surfactant may be the same or different through the different regions. In a particular embodiment, the surfactant is the same (though in different amounts) through the regions of the nanofiber structure. In certain embodiments, the surfactant is an amphiphilic block copolymer. In certain embodiments, the surfactant is an amphiphilic block copolymer comprising hydrophilic poly(ethylene oxide) (PEO) and hydrophobic poly(propylene oxide) (PPO). In certain embodiments, the surfactant comprises an amphiphilic triblock copolymer comprising a central hydrophobic PPO block flanked by two hydrophilic PEO blocks (i.e., an A-B-A triblock structure). In a particular embodiment, the surfactant comprises poloxamer 407 (Pluronic® F127).

In certain embodiments, the 3D or expanded nanofiber structure of the instant invention comprises regions with different fiber organization or alignment. In certain embodiments, the nanofiber structure comprises at least a first region and a second region, wherein the nanofiber organization or alignment of the first region is different than the nanofiber organization or alignment of the second region. In certain embodiments, the difference in nanofiber organization or alignment between regions results the pore size of the regions being different, such as at least 25%, 50%, 75%, 100%, 150%, 200%, 250%, 300%, 350%, 400%, 450%, 500% or more, larger or smaller (e.g., by volume). The nanofiber structure may comprise two or more regions. For example, the nanofiber structure may comprise 2, 3, 4, 5, 6, 7, 8, 9, 10, or more regions. The regions of the nanofiber structure can be characterized by their different nanofiber organization or alignment. In other words, each region of the nanofiber structure will have a different nanofiber organization or alignment than each adjacent region. In certain embodiments, all of the regions of the nanofiber structure have different nanofiber organization or alignment. The regions of the nanofiber structure may be arranged as layers within the nanofiber structure. The layers or regions within the nanofiber structure may have the same or different thicknesses. The layers or regions within the nanofiber structure may have the same length and width. In certain embodiments, the layers or regions within the nanofiber structure have different nanofiber organization or alignment which yields a gradient with regard to pore size. For example, in the direction of expansion, the pore size of each region or layer may increase or decrease throughout the nanofiber structure.

In accordance with the instant invention, methods for synthesizing the nanofiber structures of the instant invention comprising different nanofiber organization or alignment are provided. In certain embodiments, the method comprises electrospinning a first nanofiber region (e.g., mat or membrane) with a polymer with a first nanofiber organization or alignment and electrospinning a second nanofiber region (e.g., mat or membrane) with a polymer with a second nanofiber organization or alignment and expanding the electrospun structure to create the nanofiber structures of the instant invention comprising different nanofiber organization or alignment, wherein the first nanofiber organization or alignment is different than the second nanofiber organization or alignment. In certain embodiments, the electrospun structure is expanded using a gas foaming technique. In certain embodiments, the method comprises electrospinning two or more regions, e.g., 2, 3, 4, 5, 6, 7, 8, 9, 10, or more regions.

The polymer may be the same or different through the different regions. In a particular embodiment, the polymer is the same through the regions of the nanofiber structure. In certain embodiments, the polymer is a hydrophobic polymer. In certain embodiments, the polymer comprises polycaprolactone (PCL).

The nanofibers may have any organization or alignment. In a particular embodiment, the nanofibers may be aligned nanofibers (e.g., uniaxially, radially, vertically, or horizontally), partially aligned nanofibers (e.g., a mix of aligned and random nanofibers), random fibers, and/or entangled fibers. Aligned nanofibers may be considered different based on the direction of the alignment of nanofibers (e.g., radially vs. horizontally). Partially aligned nanofibers may be considered different from other partially aligned nanofibers based on the ratio of aligned to random nanofibers. For example, partially aligned nanofibers comprising mostly aligned nanofibers may be considered different than partially aligned nanofibers comprising mostly random nanofibers. In certain embodiments, the nanofiber structure comprises a region with aligned nanofibers and a region with random nanofibers, optionally with a region with partially aligned nanofibers. Generally, the more aligned the nanofibers of a region, the larger the pores of the region after expansion.

In certain embodiments, the 3D or expanded nanofiber structure of the instant invention comprises a compositional gradient. In certain embodiments, the nanofiber structure comprises a gradient (e.g., a concentration gradient) of at least one agent, as described hereinbelow. The nanofiber structure may comprise one or more agents, such as 2, 3, 4, 5, 6, 7, 8, 9, 10, or more agents. In certain embodiments, the nanofiber structure comprises layers or regions wherein the concentration of at least one agent in a region or layer is different than the concentration of the agent in each adjacent region or layer. In certain embodiments, the layers or regions within the nanofiber structure increase or decrease in the concentration of at least one agent. In other words, a gradient with regard to at least one agent is created. For example, in the direction of expansion, the concentration of at least one agent of each region or layer may increase or decrease throughout the nanofiber structure. The polymer may be the same or different through the different regions. In a particular embodiment, the polymer is the same through the regions of the nanofiber structure. In certain embodiments, the polymer is a hydrophobic polymer. In certain embodiments, the polymer comprises polycaprolactone (PCL).

In certain embodiments, the nanofiber structure comprises radially aligned nanofibers. In certain embodiments, the density or concentration of the agent(s) is greatest at the center of the nanofiber structure and decreases radially from the center.

In accordance with the instant invention, methods for synthesizing the nanofiber structures of the instant invention comprising a compositional gradient are provided. In certain embodiments, the method comprises electrospinning a 2D nanofiber structure (e.g., mat or membrane) comprising radially aligned nanofibers; expanding the electrospun structure; and adding the agent(s) to the center of the expanded electrospun structure, thereby creating nanofiber structures of the instant invention comprising a compositional gradient. In certain embodiments, the agent(s) are allowed to diffuse through the expanded electrospun structure (e.g., for at least 1, 2, 3, 4, 5, 6, 7, 8, 9, 10, 11, 12, 18, 24, or more hours) (e.g., radial diffusion). In certain embodiment, the method comprises electrospinning a first nanofiber region (e.g., mat or membrane) with a polymer and a first amount or concentration of at least one agent and electrospinning a second nanofiber region (e.g., mat or membrane) with a second amount or concentration of the at least one agent and expanding the electrospun structure to create the nanofiber structure of the instant invention, wherein the first amount or concentration is different than the second amount or concentration. In certain embodiments, the electrospun structure is expanded using a gas foaming technique. In certain embodiments, the polymer is a hydrophobic polymer. In certain embodiments, the polymer comprises polycaprolactone (PCL).

The nanofiber structures of the instant invention and the methods of making and/or using the same may be modified by each and any of the following features.

Generally, the synthesized 2D nanofiber structures (e.g., by electrospinning) of the instant invention are expanded, such as by a gas-foaming method. Methods for expanding nanofiber membranes or mats are described, for example, in U.S. Pat. Application Publication No. 20170296703, WO 2016/053988, WO 2019/060393, and Jiang et al. (Acta Biomater. (2017) 68:237-248), each incorporated by reference herein. In certain embodiments, the nanofiber mat is expanded into an expanded nanofiber structure by exposing the nanofiber mat to gas bubbles (e.g., gas foaming). The bubbles can be generated by chemical reactions or physical manipulations. For example, the nanofiber mat can be submerged or immersed in a bubble/gas producing chemical reaction or physical manipulation. Generally, the longer the exposure to the bubbles, the greater the thickness and porosity of the expanded nanofiber structure increases. The nanofiber mat may also be expanded within a mold (e.g., a metal, plastic, or other material that does not expand in the presence of gas bubbles) to assist in the formation of a desired shape. The nanofiber mat may be treated with air plasma prior to exposure to gas bubbles (e.g., to increase hydrophilicity).

After exposure to the bubbles, the expanded nanofiber structure may be washed and/or rinsed in water and/or a desired carrier or buffer (e.g., a pharmaceutically or biologically acceptable carrier). Trapped gas bubbles may be removed by applying a vacuum to the expanded nanofiber structure. For example, the expanded nanofiber structure may be submerged or immersed in a liquid (e.g., water and/or a desired carrier or buffer) and a vacuum may be applied to rapidly remove the gas bubbles. After expansion (e.g., after rinsing and removal of trapped gas), the expanded nanofiber structure may be placed in storage in cold solution or lyophilized and/or freeze-dried.

The gas bubbles of the instant invention can be made by any method known in the art. The bubbles may be generated, for example, by chemical reactions or by physical approaches. Electrospun nanofiber mats can be expanded in the third dimension with ordered structures using gas bubbles generated by chemical reactions in an aqueous solution (see, e.g., WO 2016/053988; WO 2019/060393; Jiang et al. (2018) Acta Biomater., 68:237-248; Jiang, et al. (2015) ACS Biomater. Sci. Eng., 1:991-1001; Jiang, et al. (2016) Adv. Healthcare Mater., 5:2993-3003; Joshi, et al. (2015) Chem. Eng. J., 275:79-88; each of the foregoing incorporated by reference herein). In a particular embodiment, the chemical reaction or physical manipulation does not damage or alter or does not substantially damage or alter the nanofibers (e.g., the nanofibers are inert within the chemical reaction and not chemically modified). As explained hereinabove, the nanofiber mat may be submerged or immersed in a liquid comprising the reagents of the bubble-generating chemical reaction. Examples of chemical reactions that generate bubbles include, without limitation:

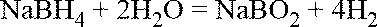

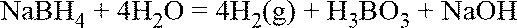

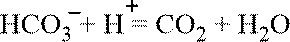

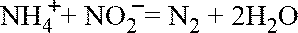

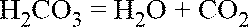

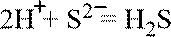

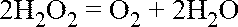

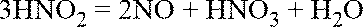

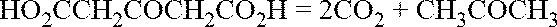

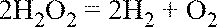

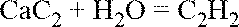

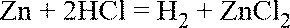

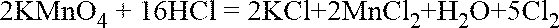

In a particular embodiment, the chemical reaction is the hydrolysis of NaBH₄ (e.g., NaBH₄ + 2H₂O = NaBO₂ + 4H₂). In a particular embodiment, CO₂ gas bubbles (generated chemically or physically) are used (e.g., for hydrophilic polymers).

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use subcritical gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber mat may be submerged or immersed in a liquid of the bubble-generating physical manipulation.

In a particular embodiment, the nanofiber mats are expanded using a subcritical or supercritical fluid or liquid (e.g., CO₂, N₂, N₂O, hydrocarbons, and fluorocarbons). In a particular embodiment, liquid CO₂ is utilized. For example, nanofiber mats may be expanded by exposing to, contacting with or being placed into (e.g., submerged or immersed) a subcritical liquid/fluid (e.g., subcritical CO₂) and then depressurized. The cycle of placing the nanofibrous structures into subcritical CO₂ and depressurizing may be performed one or more times. Generally, the more times the expansion method is used the thickness and porosity of the nanofibrous (or microfibrous) structure increases. For examples, the cycle of exposure to subcritical CO₂ and then depressurization may be performed one, two, three, four, five, six, seven, eight, nine, ten, or more times, particularly 1-10 times, 1-5 times, or 1-3 times. In a particular embodiment, the cycle of exposure to subcritical CO₂ and then depressurization is performed at least 2 times (e.g., 2-10 times, 2-5 times, 2-4 times, or 2-3 times). In a particular embodiment, the method comprises placing the nanofibrous mat and dry ice (solid CO₂) in a sealed container, allowing the dry ice to turn into liquid CO₂, and then unsealing the container to allow depressurization.

The nanofiber mat and subcritical fluid (e.g., subcritical CO₂; or solid form of subcritical fluid (e.g., dry ice)) may be contained in any suitable container (e.g., one which can withstand high pressures). For example, the subcritical fluids and the nanofiber mat may be contained within, but not limited to: chambers, vessels, reactors, chambers, and tubes. In a particular embodiment, the equipment or container used during the methods of the present invention will have a feature or component that allows control of the depressurization rate of the subcritical fluid. Depressurization of the subcritical fluid can be done using a variety of methods including but not limited to manually opening the container to decrease pressure or by using some type of equipment that can regulate the rate of depressurization of the reaction vessel.

The nanofibers of the instant invention may comprise any polymer. In a particular embodiment, the polymer is biocompatible. The polymer may be biodegradable or non-biodegradable. In a particular embodiment, the polymer is a biodegradable polymer. The polymer may by hydrophobic, hydrophilic, or amphiphilic. In a particular embodiment, the polymer is hydrophobic. In a particular embodiment, the polymer is hydrophilic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene). In a particular embodiment, the hydrophobic polymer comprises polycaprolactone (PCL).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment) - such as those listed above (e.g., gelatin/ polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium alginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA).

In a particular embodiment, the nanofiber comprises polymethacrylate, poly vinyl phenol, polyvinylchloride, cellulose, polyvinyl alcohol, polyacrylamide, poly(lactic-co-glycolic acid) (PLGA), collagen, polycaprolactone, polyurethanes, polyvinyl fluoride, polyamide, silk, nylon, polybenzimidazole, polycarbonate, polyacrylonitrile, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene gricol, polyethylene glycol, poly(ethylene-co-vinyl alcohol), polyacrylnitrile, polyvinyl pyrrolidone, polymetha-phenylene isophthalamide, gelatin, alginate, chitosan, starch, pectin, cellulose, methylcellulose, sodium polyacrylate, starch-acrylonitrile co-polymers, bioactive glass, and/or combinations of two or more polymers. Multiple polymers may be mixed to form the nanofibers. The polymers may be mixed in equal ratios or various ratios depending on the desired properties of the nanofibers. Examples of polymers and polymer combinations for nanofibers include but are not limited to PLGA-gelatin nanofibers (e.g., at a 1:1 ratio), PLGA-collagen-gelatin nanofibers (e.g., at a 1:0.5:0.5 ratio), PCL-gelatin nanofibers (e.g., at a 1:1 ratio), bioactive glass nanofibers dispersed in alginate, and PLGA short fibers dispersed in gelatin. In a particular embodiment, at least collagen and/or gelatin is used. In a particular embodiment, the polymer comprises polycaprolactone (PCL). In a particular embodiment, the polymer comprises 1) polycaprolactone (PCL) and 2) gelatin and/or collagen (e.g., at a 1:1 ratio).

In a particular embodiment, the nanofiber and/or nanofiber structure further comprises a surfactant. In a particular embodiment, the surfactant is an amphiphilic block copolymer, particularly an amphiphilic block copolymer comprising hydrophilic poly(ethylene oxide) (PEO) and hydrophobic poly(propylene oxide) (PPO). In a particular embodiment, the nanofiber comprises a poloxamer or an amphiphilic triblock copolymer comprising a central hydrophobic PPO block flanked by two hydrophilic PEO blocks (i.e., an A-B-A triblock structure). In a particular embodiment, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In a particular embodiment, the nanofiber comprises poloxamer 407 (Pluronic® F127). The amphiphilic block copolymer (e.g., poloxamer) may be added in various amounts to the polymer solution during the synthesis process (e.g., electrospinning). In a particular embodiment, 0% to 20%, particularly 0% to 10%, of the polymer solution is amphiphilic block copolymer (e.g., poloxamer). In a particular embodiment, 0.1% to 5%, particularly 0.5% to 2%, of the polymer solution is amphiphilic block copolymer (e.g., poloxamer). In a particular embodiment, the polymer solution comprises about 5% to about 20%, or about 10% polymer (e.g., PCL).

In a particular embodiment, the nanofibers and/or nanofiber structures are coated with additional materials to enhance their properties. For example, the nanofibers and/or nanofiber structure may be coated with collagen, a proteoglycans, elastin, a glycosaminoglycan (e.g., hyaluronic acid, heparin, chondroitin sulfate, or keratan sulfate), gelatin, alginate, chitosan, collagen, starch, pectin, cellulose, methylcellulose, sodium polyacrylate, and/or starch-acrylonitrile co-polymers. In a particular embodiment, the nanofibers and/or nanofiber structure comprise a material that enhances the nanofiber structure’s ability to absorb fluids, particularly aqueous solutions (e.g., blood; e.g., for use of hemostatic materials). In a particular embodiment, the nanofibers and/or nanofiber structure are coated with the material which enhances the absorption properties. In a particular embodiment, the nanofibers and/or nanofiber structure comprise a material that enhances the nanofiber structure’s ability to recover its shape and/or structure after compression (e.g., compression of the thickness of the expended nanofiber structure (e.g., into a 2D membrane or structure)). In a particular embodiment, the nanofibers and/or nanofiber structure are coated with the material which enhances the recovery properties. In a particular embodiment, the nanofibers and/or nanofiber structure comprises and/or is coated with gelatin, particularly to enhance or increase the 3D nanofiber structure’s ability to absorb fluids and shape recovery after compression and/or reduce or slow diffusion of agents within the nanofiber structure.

The term “coat” refers to a layer of a substance/material on the surface of a structure. Coatings may, but need not, also impregnate the nanofibers and/or nanofiber structure. Further, while a coating may cover 100% of the nanofiber and/or nanofiber structure, a coating may also cover less than 100% of the surface of the nanofiber and/or nanofiber structure (e.g., at least about 75%, at least about 80%, at least about 85%, at least about 90%, at least about 95%, at least about 98%, or more of the surface may be coated). Materials which enhance the absorption properties of the nanofibers and/or nanofiber structure include, without limitation: gelatin, alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In a particular embodiment, the material is a hydrogel (e.g., a polymer matrix able to retain water, particularly large amounts of water, in a swollen state). In a particular embodiment, the material is gelatin. In a particular embodiment, the nanofibers and/or nanofiber structures are coated with about 0.05% to about 10% coating material (e.g., gelatin), particularly about 0.1% to about 10% coating material (e.g., gelatin) or about 0.1% to about 1% coating material (e.g., gelatin). In a particular embodiment, the material (e.g., hydrogel) is crosslinked (e.g., by UV crosslinking).

In a particular embodiment, the nanofibers and/or nanofiber structure are mineralized (e.g., comprise minerals and/or coated with minerals). Mineralization, for example, with hydroxyapatite, can enhance the adhesion of osteogenic precursor cells in vitro and in vivo (Duan, et al., Biomacromolecules (2017) 18:2080-2089). In a particular embodiment, the nanofibers and/or nanofiber structure are coated with Ca, P, and O. In a particular embodiment, the nanofibers and/or nanofiber structure are coated with hydroxyapatite, fluorapatite, or chlorapatite, particularly hydroxyapatite. In a particular embodiment, the nanofibers and/or nanofiber structure are immersed in simulated body fluid (SBF) for the mineralization (e.g., a solution comprising NaCl, CaCl₂, NaH₂PO₄, and NaHCO₃.

As stated herein, the nanofiber structures of the instant invention may be crosslinked and/or thermally treated (e.g., to enhance their stability). Crosslinking may be done using a variety of techniques including thermal crosslinking, chemical crosslinking, UV-crosslinking, and photo-crosslinking. For example, the nanofiber structures of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In a particular embodiment, the crosslinker is glutaraldehyde. In a particular embodiment, the nanofiber structure is thermally treated (e.g., at a temperature close to, but below, the melting point of the nanofibers; e.g., about 50° C.).

After synthesis, the nanofiber structures may be washed or rinsed in water and/or a desired carrier or buffer (e.g., a pharmaceutically or biologically acceptable carrier). The nanofiber structures may also be stored in a cold solution, lyophilized and/or freeze-dried. The nanofiber structures may also be physically manipulated such as compressing and/or shaping or trimming of the nanofiber structure (e.g., to achieve a desired shape).

The nanofiber structures of the instant invention may also be sterilized. For example, the nanofiber structures can be sterilized using various methods (e.g., by treating with ethylene oxide gas, gamma irradiation, or 70% ethanol).

The nanofiber structures of the instant invention may comprise and/or encapsulate cells or tissue. In a particular embodiment, the cells are autologous to the subject to be treated with the nanofiber structure. The nanofiber structures may comprise and/or encapsulate any cell type. Cell types include, without limitation: embryonic stem cells, adult stem cells, bone marrow stem cells, induced pluripotent stem cells, progenitor cells (e.g., neural progenitor cells), embryonic like stem cells, mesenchymal stem cells, bone marrow mesenchymal stem cells, CAR-T cells, immune cells (including but not limited to T cells, B cells, NK cells, macrophages, neutrophils, dendritic cells and modified forms of these cells and various combinations thereof), cell based vaccines, and cell lines expressing desired therapeutic proteins and/or genes. In a particular embodiment, the cells comprise stem cells. In a particular embodiment, the cells comprise bone marrow stem cells. In a particular embodiment, the cells comprise dermal fibroblasts. In a particular embodiment, the nanofiber structure comprises and/or encapsulates cell spheroids. In a particular embodiment, the nanofiber structure comprises and/or encapsulates tissue samples (e.g., minced tissue), such as skin tissue samples or bone samples. The cells or tissue may be cultured within the nanofiber structures (e.g., the cells or tissue may be cultured for sufficient time to allow for growth within and/or infiltration into the nanofiber structure). For example, the cells or tissue may be cultured in the nanofiber structure for 1, 2, 3, 4, 5, 6, 7, 8, 9, 10, 11, 12, 13, 14, 15, 16, 17, 18, 19, 20, or more days. In certain embodiments, the cells or tissue may be cultured within the nanofiber structures in differentiation media. In a particular embodiment, the cells or tissue may be seeded into the nanofiber structure under a vacuum. In a particular embodiment, the nanofiber structure is in solution with the cells or tissue and the nanofiber structure is further expanded and/or a vacuum is applied, thereby resulting in the seeding of the cells or tissue within the nanofiber structure.

The nanofiber structures of the instant invention may comprise or encapsulate at least one agent, particularly a bioactive agent, biologic, cell based therapy, tissue based therapy, and/or drug. In a particular embodiment, the nanofiber structure is coated with the agent and/or the agent is contained within the nanofiber structure and/or a coating of the nanofibers and/or nanofiber structure. In a particular embodiment, the agent is hydrophilic. The agent may be added to the nanofiber structure during synthesis and/or after synthesis. The agent may be conjugated (e.g., directly or via a linker) to the nanofiber structure and/or coating material, encapsulated by the nanofiber structure, and/or coated on the nanofiber structure (e.g., with, underneath, and/or on top of the coating of the nanofiber structure’s, if present). In a particular embodiment, the agent is not directly conjugated to the nanofiber structure (e.g., it is encapsulated). In a particular embodiment, the agent is conjugated or linked to the nanofiber structure (e.g., surface conjugation or coating). In a particular embodiment, the agents are administered with but not incorporated into the nanofiber structures.

Biologics include but are not limited to proteins, peptides, antibodies, antibody fragments, nucleic acid, DNA, RNA, and other known biologic substances, particularly those that have therapeutic use. In a particular embodiment, the agent is a drug or therapeutic agent (e.g., a small molecule) (e.g., analgesic, growth factor, anti-inflammatory, signaling molecule, cytokine, antimicrobial (e.g., antibacterial, antibiotic, antiviral, and/or antifungal), hemostatic agent (e.g., blood clotting agent, factor, or protein), pain medications (e.g., anesthetics), etc.). In a particular embodiment, the agent enhances tissue regeneration, tissue growth, and wound healing (e.g., growth factors). In a particular embodiment, the agent treats/prevents infections (e.g., antimicrobials such as antibacterials, antivirals and/or antifungals). In a particular embodiment, the agent is an antimicrobial, particularly an antibacterial. In a particular embodiment, the agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFβ, TGFβ3), nerve growth factors (NGF), neurotrophic factors, stromal derived factor-1 (SDF-1), granulocyte-macrophage colony-stimulating factor (GM-CSF), granulocyte-colony stimulating factor (G-CSF), erythropoietin (EPO), glial cell-derived neurotrophic factors (GDNF), hepatocyte growth factors (HGF), keratinocyte growth factors (KGF), and/or growth factor mimicking peptides (e.g., VEGF mimicking peptides). In a particular embodiment, the growth factor is bFGF. Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, I-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In a particular embodiment, the agent is a blood clotting factor such as thrombin or fibrinogen. In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 1). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 1). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antibacterial. Examples of antimicrobials include, without limitation, antibiotics such as beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin); silver containing compounds (e.g., silver ions, silver nitrate, silver nanoparticles, colloidal silver, etc.), gallium containing compounds (e.g., gallium ions, gallium nitrate, gallium nanoparticles, colloidal gallium, etc.), and antimicrobial peptides. Examples of antifungals include, without limitation, amphotericin B, pyrimethamine, thiazoles, allylamines, flucytosine, caspofungin acetate, fluconazole, griseofulvin, terbinafine, amorolfine, imidazoles, triazoles (e.g., voriconazole), flutrimazole, cilofungin, echinocandines, pneumocandin omoconazole terconazole, nystatin, natamycin, griseofulvin, ciclopirox, naftifine, and itraconazole. In a particular embodiment, the antimicrobial is an antibiotic. In a particular embodiment, the antimicrobial is an antimicrobial peptide. In a particular embodiment, the nanofiber and/or nanofiber structure comprises an antimicrobial peptide and at least one other antimicrobial (e.g., antibiotic). Antimicrobial peptides may be therapeutically effective against one or more bacteria. Examples of antimicrobial peptides are provided in the Antimicrobial Peptide Database (aps.unmc.edu/AP/main.php). Examples of antimicrobial peptides are also disclosed in U.S. Pat. No. 7,465,784, U.S. Pat. No. 9,580,472, U.S. Pat. No. 10,144,767, U.S. Pat. Application Publication No. 20090156499, U.S. Pat. Application Publication No. 20150259382, U.S. Pat. Application Publication No. 20140303069, and PCT/US2019/039792, each incorporated by reference herein. In a particular embodiment, the antimicrobial peptide has fewer than about 50 amino acids, fewer than about 25 amino acids, fewer than about 20 amino acids, fewer than about 17 amino acids, fewer than about 15 amino acids, fewer than 12 amino acids, fewer than 10 amino acids, or fewer than 9 amino acids. In a particular embodiment, the antimicrobial peptide has more than about 6 amino acids, particularly more than about 7 amino acids.

The nanofiber structures of the present invention may also be modified with targeting moieties (e.g., to enhance delivery to specific sites within the body (e.g., tissue types, disease areas, etc.)). Examples of targeting moieties include but are not limited to peptides, proteins, antibodies, antibody fragments, and small molecules. In a particular embodiment, a nanofiber of the structure is linked to a targeting ligand. A targeting ligand is a compound that specifically or preferentially binds to a specific type of tissue or cell type. For example, a targeting ligand may be used for engagement or binding of a target cell (e.g., a surface marker or receptor). In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type. The targeting ligand may be linked directly to the nanofiber or structure or via a linker. Generally, the linker is a chemical moiety comprising a covalent bond or a chain of atoms that covalently attaches the ligand to the polymer or surfactant. The linker can be linked to any synthetically feasible position of the ligand and the polymer or surfactant. Exemplary linkers may comprise at least one optionally substituted; saturated or unsaturated; linear, branched or cyclic aliphatic group, an alkyl group, or an optionally substituted aryl group. The linker may be a lower alkyl or aliphatic. The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions. The nanofiber structures of the instant invention may comprise targeted and/or non-targeted nanofibers. In a particular embodiment, the molar ratio of targeted and non-targeted nanofibers in the structure of the instant invention is from about 0.001 to 100%, about 1% to about 99%, about 5% to about 95%, about 10% to about 90%, about 25% to about 75%, about 30% to about 60%, or about 40%.

The nanofiber structures of the instant invention can be used to create tissue architectures for a variety of application including, without limitation: wound healing, tissue engineering, tissue growth, tissue repair, tissue regeneration, and engineering 3D in vitro tissue models. Some examples of uses for the nanofiber structures of the instant invention include, but are not limited to: use as tissue structures (in vitro or in vivo), hemostatic bandages, tissue repair structures, and tissue regeneration structures. Unlike other expanded nanofiber structures, nanofiber structures described herein can allow for cell infiltration from both the top/bottom and sides, thereby providing enhanced properties for use in cell growth, tissue repair, tissue regeneration, and even non-biomedical uses such as filtrations systems/filters.

The nanofiber structures can also be combined with a variety of hydrogels or biological matrices/cues to form 3D hybrid structures that can release biologically functional agents. The tissue constructs can be used for regeneration of many tissue defects (e.g., skin, bone, cartilage) and healing of various wounds (e.g., injuries, diabetic wounds, venous ulcer, pressure ulcer, burns). The nanofiber structures may be used ex vivo to generate tissue or tissue constructs/models. The nanofiber structures may also be used in vivo in patients (e.g., human or animal) for the treatment of various diseases, disorders, and wounds. In a particular embodiment, the nanofiber structure stimulates the growth of existing tissue and/or repair of a wound or defect when applied in vivo. The nanofiber structures can be used for engineering, growing, and/or regeneration of a variety of tissues including but not limited to skin, bone, cartilage, muscle, nervous tissue, and organs (or portions thereof).

In accordance with the instant invention, the nanofiber structures may be used in inducing and/or improving/enhancing wound healing and inducing and/or improving/enhancing tissue regeneration. The nanofiber structures of the present invention can be used for the treatment, inhibition, and/or prevention of any injury or wound. In a particular embodiment, the method comprises administering nanofiber structure comprising an agent and/or cell as described herein. Nanofiber structures of the instant invention can be loaded with different cell types as necessary for regeneration of various tissues. In a particular embodiment, the nanofiber structure comprises blood clotting factors (e.g., for accelerating blood clot formation and/or preventing blood loss). For example, the nanofiber structure can be used to induce, improve, or enhance wound healing associated with surgery (including non-elective (e.g., emergency) surgical procedures or elective surgical procedures). Elective surgical procedures include, without limitation: liver resection, partial nephrectomy, cholecystectomy, vascular suture line reinforcement and neurosurgical procedures. Non-elective surgical procedures include, without limitation: severe epistaxis, splenic injury, liver fracture, cavitary wounds, minor cuts, punctures, gunshot wounds, and shrapnel wounds. The nanofiber structure of the present invention can also be incorporated into delivery devices that allow for their injection/delivery directly into a desired location (e.g., a wound). The nanofiber structures also may be delivered directly into a cavity (such as the peritoneal cavity) (e.g., using a pressurized cannula).

In accordance with the instant invention, the nanofiber structures of the present invention can be used to treat and/or prevent a variety of diseases and disorders. Examples of diseases and/or disorders include but are not limited to wounds, ulcers, infections, hemorrhage, tissue injury, tissue defects, tissue damage, bone fractures, bone degeneration, cartilage damage, cancer (e.g., the use of docetaxel and curcumin for the treatment of colorectal cancer (Fan, et al., Sci. Rep. (2016) 6:28373)), neurologic diseases (e.g., Alzheimer’s and Parkinson’s), ischemic diseases, inflammatory diseases and disorders, heart disease, myocardial infarction, and stroke. Methods for inducing and/or improving/enhancing wound healing in a subject are also encompassed by the instant invention. Methods of inducing and/or improving/enhancing tissue regeneration (e.g., blood vessel growth, neural tissue regeneration, and bone and/or cartilage regeneration) in a subject are also encompassed by the instant invention. Methods of inducing and/or improving/enhancing hemostasis in a subject are also encompassed by the instant invention. The methods of the instant invention comprise administering or applying nanofiber structures of the instant invention to the subject (e.g., at or in a wound). In a particular embodiment, the method comprises administering nanofiber structures comprising an agent and/or cell as described herein. Nanofiber structures of the instant invention can be loaded with different cell types as necessary for regeneration of various tissues. In a particular embodiment, the nanofiber structures comprise blood clotting factors (e.g., for accelerating blood clot formation and/or preventing blood loss). In a particular embodiment, the method comprises administering nanofiber structures to the subject and an agent as described herein (i.e., the agent is not contained within the nanofiber structure). When administered separately, the nanofiber structure may be administered simultaneously and/or sequentially with the agent. The methods may comprise the administration of one or more nanofiber structure. When more than one nanofiber structure is administered, the nanofiber structures may be administered simultaneously and/or sequentially.

The nanofiber structures can also be used to expand and increase cell numbers (e.g., stem cell numbers) in culture. In a particular embodiment, microtissues can be grown in situ by prolonged culture of cell laden nanofiber structures (e.g., in confined microfluidic channel devices). These microtissues are injectable or transplantable into a tissue defect to promote wound healing in a subject (e.g., the nanofiber structures comprise autologous cells).

The nanofiber structures may also be employed for cell detection, separation, and/or isolation of cell populations in a mixture. For example, structures conjugated to specific antibodies can be used for the isolation, separation, and/or expansion of different cell types from their mixtures (Custodio, et al., Biomaterials (2015) 43:23-31). Further, nanofiber structures can be used for the in vitro adhesion, proliferation, and/or maturation of chondrocytes as well as in vivo cartilage formation and osteochondral repair induced by nanofiber structures when together with chondrocytes (Liu, et al., Nat. Mater. (2011) 10:398-406).

The nanofiber structures of the present invention may be administered by any method. The nanofiber structures described herein may be administered to a subject or a patient as a pharmaceutical composition. The compositions of the instant invention comprise nanofiber structure and a pharmaceutically acceptable carrier. The term “patient” as used herein refers to human or animal subjects. These compositions may be employed therapeutically, under the guidance of a physician.

The compositions of the instant invention may be conveniently formulated for administration with any pharmaceutically acceptable carrier(s). For example, the agents may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. Except insofar as any conventional media or agent is incompatible with the agents to be administered, its use in the pharmaceutical preparation is contemplated.

Compositions of the instant invention may be administered by any method. For example, the compositions of the instant invention can be administered, without limitation, parenterally, subcutaneously, orally, topically (ex. using a cream or spray), pulmonarily, rectally, vaginally, intravenously, intraperitoneally, intrathecally, intracerbrally, epidurally, intramuscularly, intradermally, intratumoral, intracarotidly, or by direct injection (e.g., a localized injection into a specific tissue or organ). Selection of a suitable pharmaceutical preparation will also depend upon the mode of administration chosen. For example, the compositions of the invention may be administered parenterally. In this instance, a pharmaceutical preparation comprises the nanofiber structures dispersed in a medium that is compatible with the parenteral injection.

Pharmaceutical compositions containing an agent of the present invention as the active ingredient in intimate admixture with a pharmaceutically acceptable carrier can be prepared according to conventional pharmaceutical compounding techniques.

In a particular embodiment of the instant invention, methods for modulating (increasing) hemostasis; inhibiting blood or cartilage loss; and/or treating hemorrhage are provided. In a particular embodiment, the method comprises administering the nanofiber structure to the wound or site of bleeding. In a particular embodiment, the nanofiber structures comprise a blood clotting factor such as thrombin and/or fibrinogen.

In a particular embodiment of the instant invention, methods for stimulating bone and/or cartilage regeneration and/or treating bone and/or cartilage loss are provided. In a particular embodiment, the method comprises administering the nanofiber structures to the site of bone and/or cartilage loss. In a particular embodiment, the site of bone and/or cartilage loss is periodontal. In a particular embodiment, the nanofiber structures are mineralized. In a particular embodiment, the nanofiber structures comprise a bone growth stimulating growth factor such as a bone morphogenic protein or fragment or analog thereof. In a particular embodiment, the agent is a bone morphogenetic protein (e.g., BMP-2, BMP-7, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 1). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 1). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

In accordance with the instant invention, antimicrobial (e.g., antibiotic)-loaded nanofiber structures are provided. In a particular embodiment, the antimicrobial (e.g., antibiotic)-loaded nanofiber structure is in the form of a wound dressing. The antimicrobial (e.g., antibiotic)-loaded nanofiber structure may be in any form including, without limitation, a wound dressing, bandage, gauze, covering, suture, thread, ligature, hemostasis material, or coating for biomedical device or implant. In a particular embodiment, the antimicrobial (e.g., antibiotic)-loaded nanofiber structure is in a wound dressing.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHC1, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington’s Pharmaceutical Sciences” by E.W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject’s body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following example illustrates certain embodiments of the invention. It is not intended to limit the invention in any way.

### EXAMPLE

Herein, the conversion of 2D electrospun poly(ε-caprolactone) nanofiber membranes into biomimetic, stem-cell regulating, 3D hierarchical assemblies with structural and compositional gradients is reported. The present study is based, in part, on: i) a gas-foaming technology expanding 2D electrospun nanofiber mats in the third dimension; ii) transformation of 2D nanofiber membranes into 3D objects with hierarchical structure and controlled fiber alignment; iii) incorporation of a surfactant (e.g., Pluronic® F-127) to nanofibers to significantly increases hydrophilicity and subsequent gas bubble stability, resulting in a faster expansion rate of membranes; and/or iv) gelatin-coated expanded nanofiber scaffolds are superelastic and compressible.

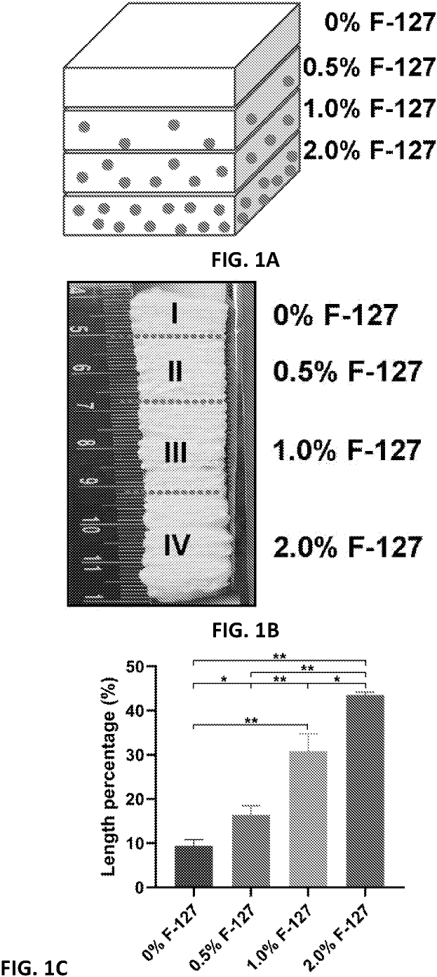

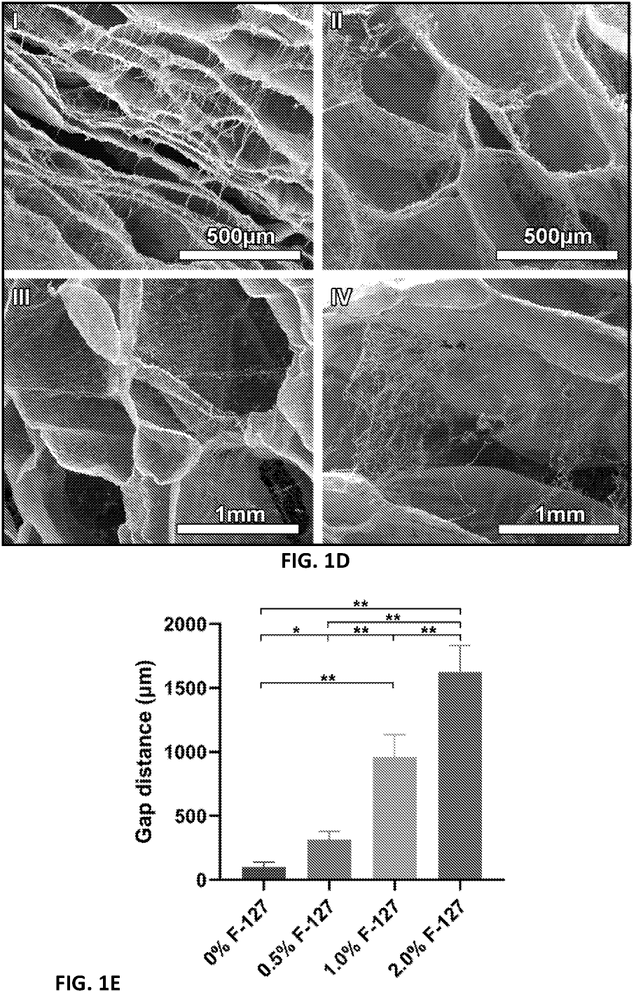

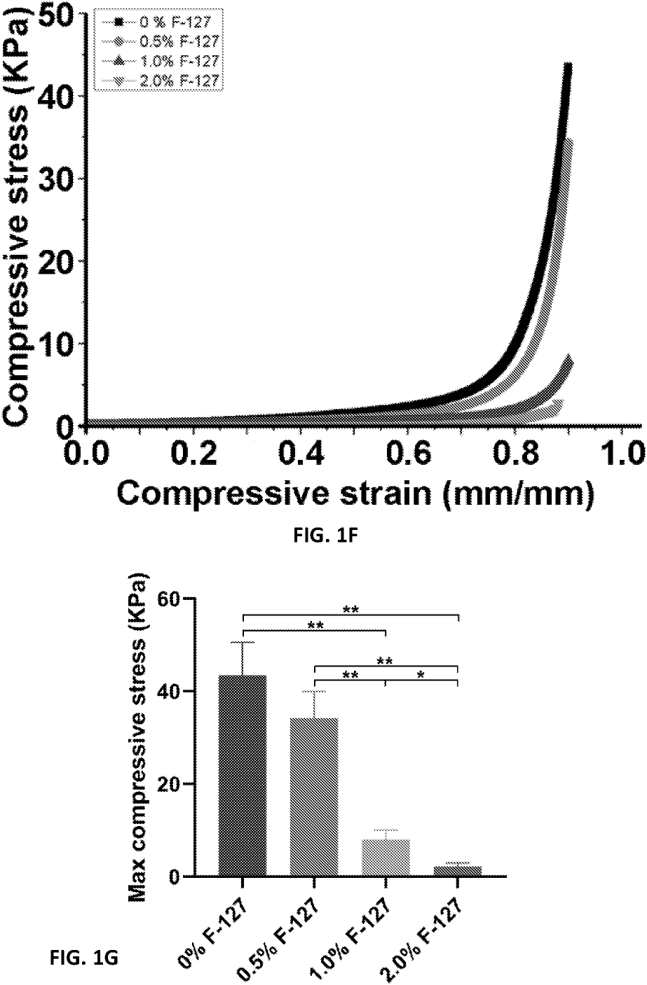

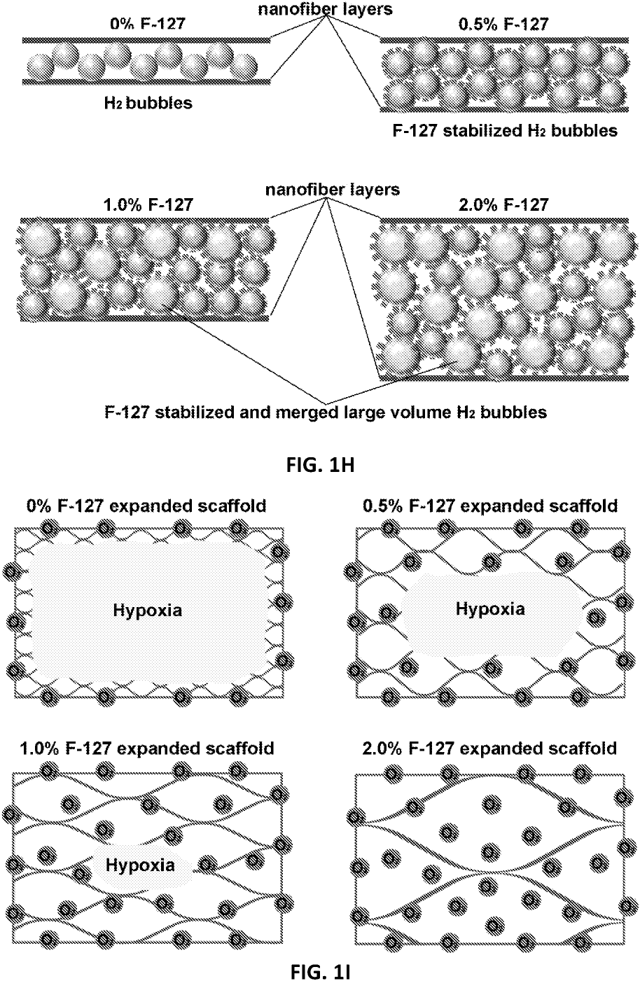

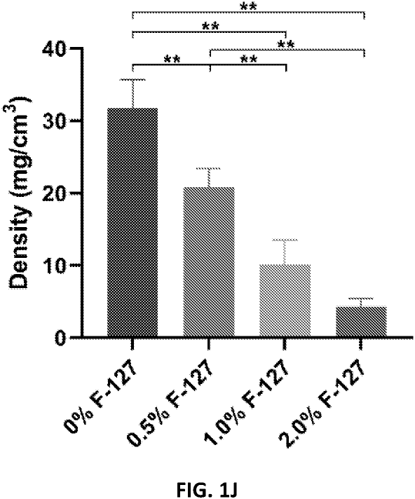

First, the fabrication of 3D nanofiber scaffolds with structural gradients in pore sizes was demonstrated. By decreasing the amounts of Pluronic® F-127 incorporated to nanofibers in each successive layer, 2D nanofiber membranes can be converted into 3D assemblies with gradient in pore sizes after the gas-foaming expansion process as each successive layer expanded less than the previous layer. Briefly, 1-mm thick PCL/Pluronic® F-127 nanofiber membranes were prepared by sequential deposition of PCL nanofibers with incorporation of 2%, 1%, 0.5% and 0% Pluronic® F-127 from bottom to top on the rotating mandrel as illustrated in FIG. 1A. FIG. 1B shows a typical photograph of an expanded 3D nanofiber scaffold with a pore size gradient following the gas-foaming expansion (the hydrolysis of NaBH₄ (e.g., NaBH₄ + 2H₂O = NaBO₂ + 4H₂)). The length percentage of each region after expansion significantly increased with increasing the amount of blended Pluronic® F-127 from 0 to 2% (FIG. 1C). FIG. 1D shows SEM images of each distinct regions, revealing a gradual increase in pore sizes with increased blended Pluronic® F-127 content, resulting in a gradual decrease of density (FIG. 1J). Gap distances in each region ranged from 98.5 ± 40.3 µm, to 271.2 ± 64.5 µm, to 940.7 ± 177.3 µm, and further to 1711.9 ± 225.8 µm which corresponded to 0%, 0.5%, 1% and 2% of Pluronic® F-127 blend. Regions blended with more Pluronic® F-127 had lower maximum compressive stress (FIG. 1F and FIG. 1G). Without being bound by theory, Pluronic® F-127 reduces the surface tension and occupies the interface between the gas phase and liquid phase, and inhibits the diffusion of H₂ gas, thereby stabilizing H₂ gas bubbles and accelerating the expansion of nanofiber membranes. The formation of 3D nanofiber scaffolds with gradient in pore sizes were due to regionally different Pluronic® F-127 concentrations and the expansion rate of the regions with incorporation of less Pluronic® F-127 was slower compared to the regions containing more Pluronic® F-127 (FIG. 1H). The 3D nanofiber scaffolds with gradient in pore sizes can be used to create a gradient hypoxic environment that plays an important role in regulating stem cell responses. The hypoxia level can gradually decrease by increasing the pore sizes of regions containing 0 to 2% Pluronic® F-127 (FIG. 11). The principle of hypoxia gradients rendered by such 3D scaffolds is similar to the rolling of scaffold strip and stacking of paper scaffolds, which is mainly due to the slower diffusion of oxygen in the denser region. By comparison, the 3D scaffolds developed herein are more physiologically relevant due to the ECM-mimicking nanofibrous matrix.

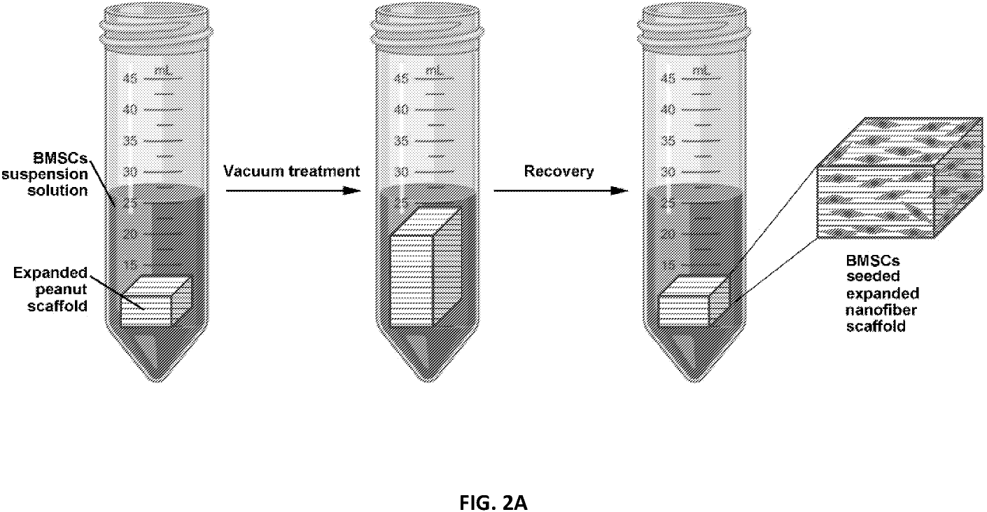

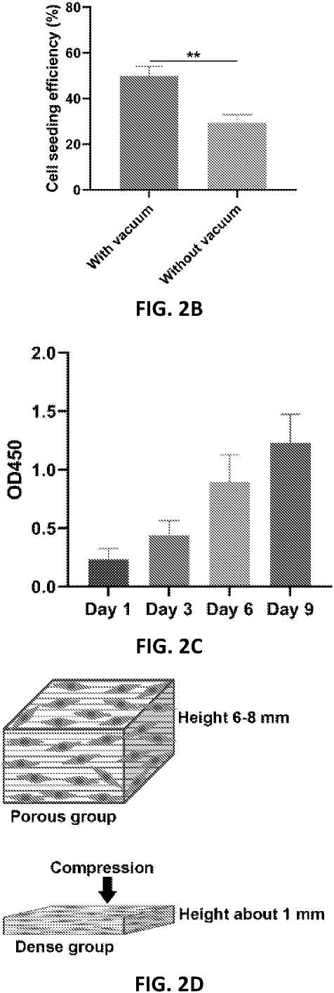

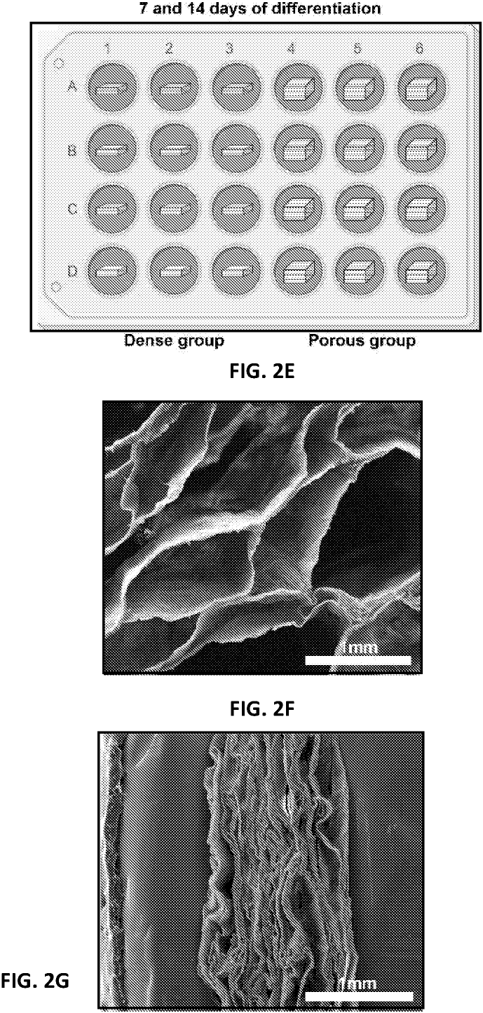

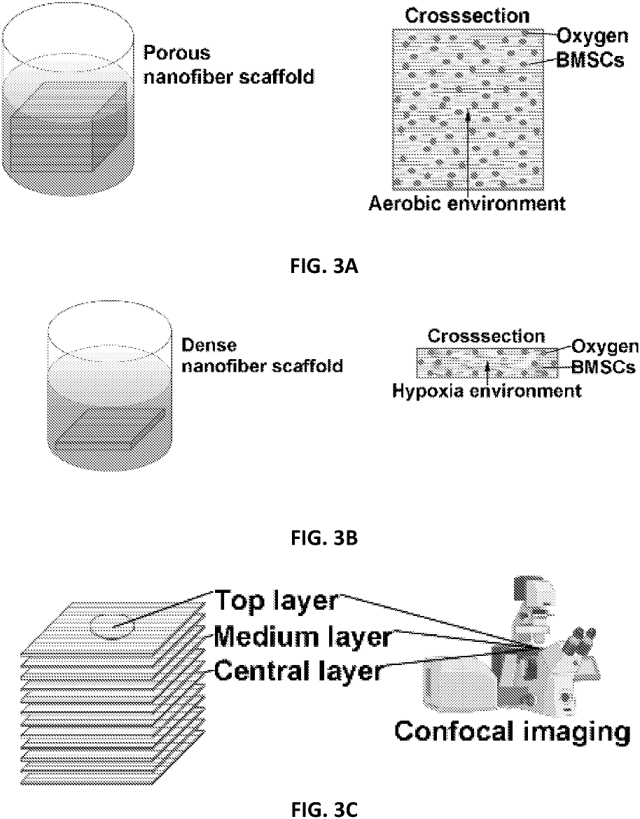

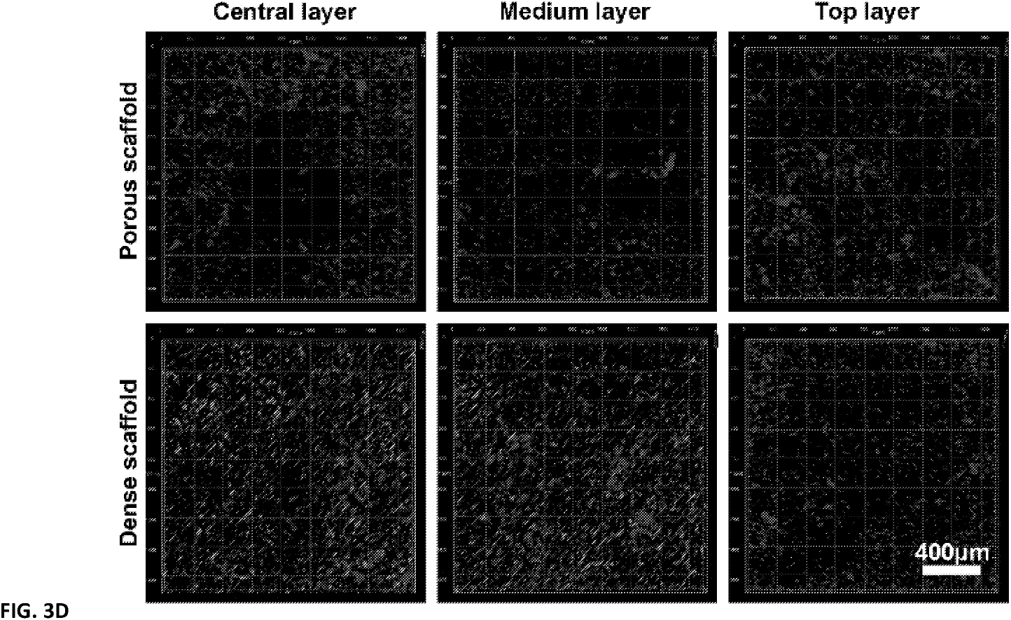

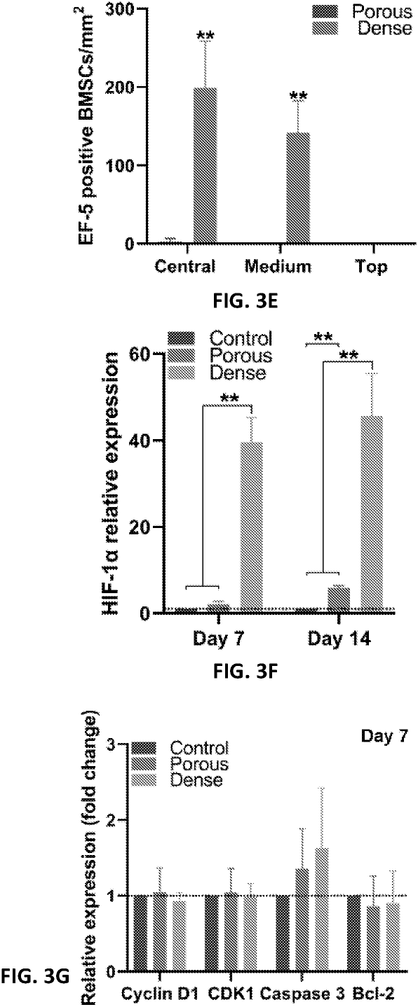

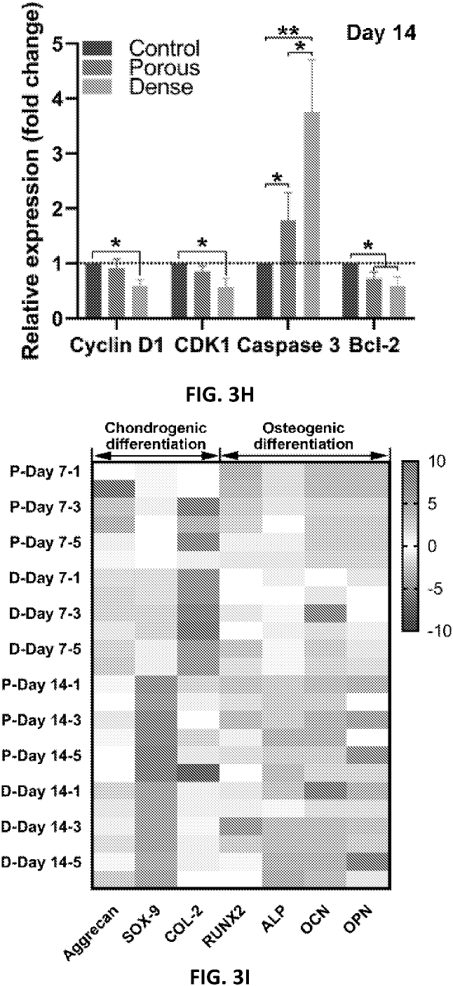

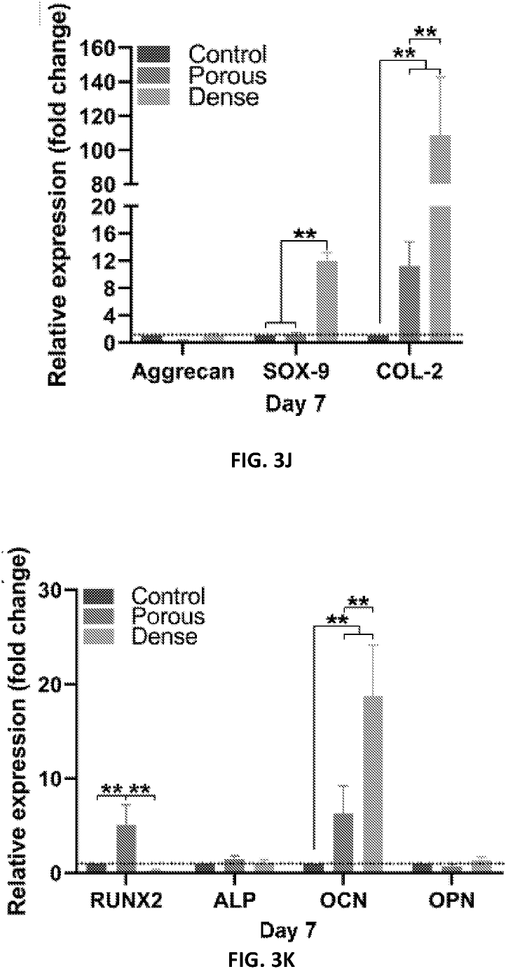

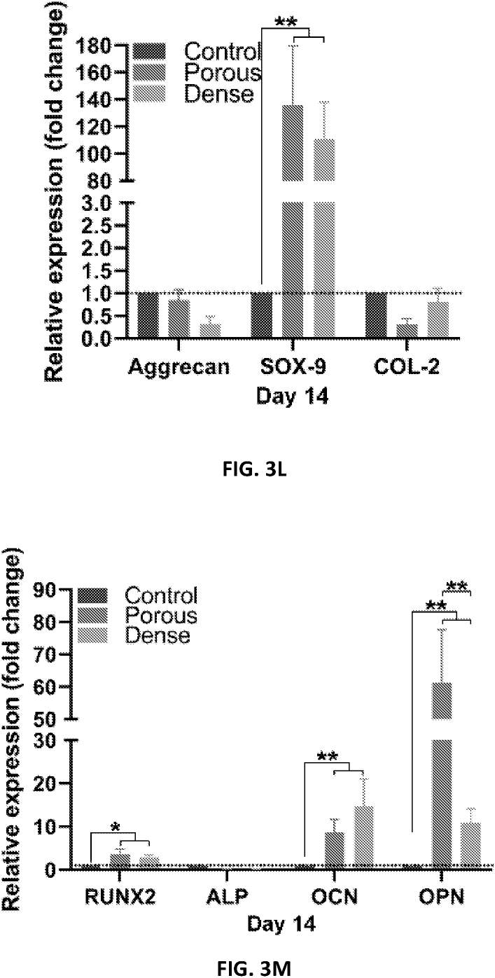

To demonstrate the effect of pore size on the cell response, bone marrow stem cells (BMSCs) were seeded onto expanded, rectangular nanofiber scaffolds (10 mm × 10 mm × 6-8 mm). To enhance the cell seeding efficiency, the rectangular-shaped scaffolds were immersed in the cell suspension and further expanded to allow cells to penetrate into scaffolds by vacuum for 10 seconds, thus facilitating increased cell penetration into scaffolds (FIG. 2A). Using this method allows cell seeding efficiency to reach nearly 50%, which is significantly higher than traditional seeding methods without applying vacuum (often < 30%) (FIG. 2B). BMSCs-seeded scaffolds were incubated in the proliferation medium for 9 days and subsequently divided into two groups (porous and dense scaffolds) (FIG. 2C). The porous group maintained the heights of 6-8 mm, and the dense group was compressed to the heights of around 1 mm (FIG. 2D and FIGS. 3A and 3B). These two groups of scaffolds were further incubated in the chondrogenic and osteogenic differentiation medium for 7 and 14 days (FIG. 2E). FIGS. 2F and 2G show SEM images of the cross sections of the 6-8 mm and 1-mm high scaffolds, indicating the porous and dense structures, which corresponded to the regions containing 1% and 0% Pluronic® F-127, respectively. The middle of the top, medium, and central layers of porous and dense scaffolds were used for microscopic examination (FIG. 3C).

FIGS. 3D and 3E show that BMSCs in the top layers of the porous and dense nanofiber scaffolds had similar expression patterns of (2-(2-Nitro-1H-imidazol 1-yl)-N-(2,2,3,3,3-pentafluoropropyl) acetamide) (EF-5), a cellular hypoxic indicator (Rodenhizer, et al., Nat. Mater. (2016) 15:227-234). In contrast, the medium and central layers of the dense scaffolds had a significantly higher number of EF-5 positive cells than the medium and central layers of the porous scaffolds. Compared to the BMSCs on culture plates and porous scaffolds, the BMSCs on the dense scaffolds showed a significant increase in the expression of hypoxia-inducible factor 1-alpha (HIF-1α), a major transcriptional regulator of cellular and developmental responses to hypoxia (FIG. 3F) (Semenza, G.L., Cell (2012) 148:399-408). These results indicate that a hypoxic environment can be established in the dense region of expanded 3D nanofiber scaffold with gradient in pore sizes. In addition, BMSCs on both porous and dense groups showed the similar expression of cell cycle-related genes (Cyclin D1 and CDK1), apoptotic (Caspase-3), and anti-apoptotic related genes (Bcl-2) after incubation for 7 days in the chondrogenic differentiation medium (FIG. 3G). However, after incubation for 14 days, BMSCs in the dense group showed a decrease in the Cyclin D1 and CDK 1 expression compared to the control (FIG. 3H). The expression of Caspase-3 was significantly increased in both porous and dense groups compared to the control, while the expression of Bcl-2 was decreased (FIG. 3H). These results indicate that a long-term hypoxic environment may inhibit BMSCs proliferation and promote BMSCs apoptosis.

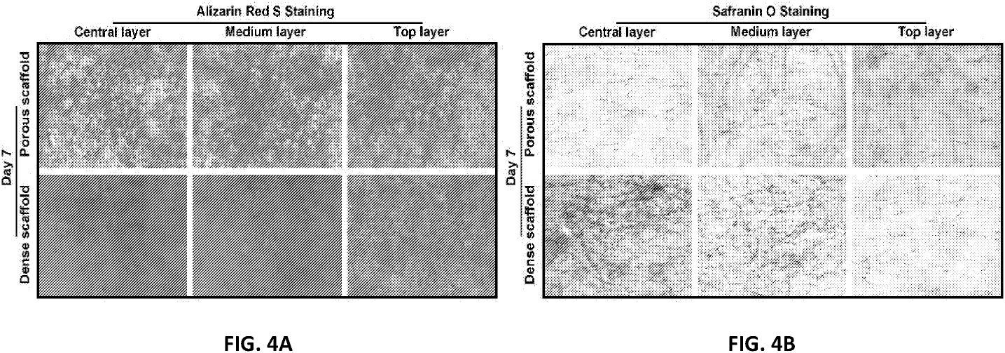

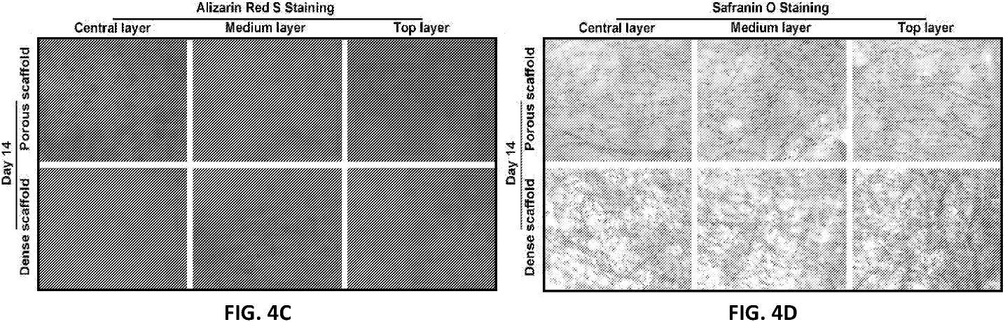

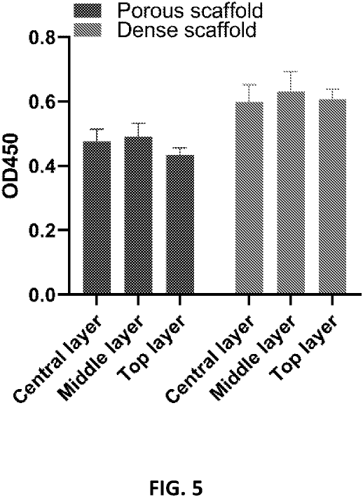

Gene expression heatmapping showed the upregulation and downregulation of chondrogenic and osteogenic differentiation-related genes of BMSCs on porous and dense scaffolds (FIG. 31). On one hand, after 7 days of chondrogenic differentiation, relative SOX-9 and type 2 collagen expression of BMSCs on dense scaffolds was dramatically increased relative to cells on the porous scaffolds (FIG. 3J). Relative SOX-9 expression of BMSCs on both porous and dense scaffolds was higher than the control after 14 days of chondrogenic differentiation (FIG. 3L). The safranin O staining also revealed that more proteoglycans were detected in dense nanofiber scaffolds on both Day 7 and Day 14, with the highest concentrations in the central layers having the highest level of hypoxia (FIGS. 4B and 4D). On the other hand, after 7 days of osteogenic differentiation, relative RUNX2 expression of BMSCs on porous nanofiber scaffolds was higher than those on both dense scaffolds and controls. In contrast, the OCN expression of BMSCs on the dense scaffolds was higher than those on both porous scaffolds and controls (FIG. 3K). After 14 days of osteogenic differentiation, no difference was observed in the expression of RUNX-2, OCN, and OPN of BMSCs on porous or dense scaffolds, but cells on both scaffolds expressed each gene higher than the controls (FIG. 3M). Although there was no trend in expression of osteogenic differentiation related genes, the alizarin Red S staining results indicate a mass of deposited calcium in the central and intermediate layers of the dense scaffolds after 7 days of osteogenic differentiation (FIG. 4A). However, no difference was observed in the calcium deposition between the porous and dense scaffolds after 14 days of osteogenic differentiation (FIG. 4C). Differences in proteoglycans and deposited calcium were not caused by the disparity of cell numbers on scaffolds (FIG. 5).

Taken together, these results indicate that dense nanofiber scaffolds can provide a hypoxic environment and promote chondrogenic differentiation of BMSCs while exerting marginal influence on osteogenic differentiation better than porous scaffolds. The implantation of BMSCs after osteogenic differentiation or hypoxia preconditioned BMSCs can enhance bone regeneration in vivo compared to undifferentiated cells (Jordahl, et al., Adv. Mater. (2018) 30:e1707196; Zhang, et al., Int. J. Biol. Sci. (2018) 14:449-460). In addition, chondrogenic differentiation of BMSCs can recapitulate the bone development and endochondral ossification for bone regeneration (Dennis, et al., Tissue Eng. Part B Rev. (2015) 21:247-266; Sheehy, et al., Mater. Today Bio. (2019) 3:100009). Therefore, the 3D nanofiber scaffolds with gradients in pore sizes developed herein can be used in engineering heterogeneous tissue constructs for osteochondral repair. After BMSCs seeding and proliferation, expanded scaffolds can be compressed to form a tunable hypoxic environment for regulating chondrogenic differentiation by modulating gene expression, which will offer increased control in cartilage and bone repair. Such nanofiber assemblies can also be used as a biomimetic hypoxia platform for cell biology study.

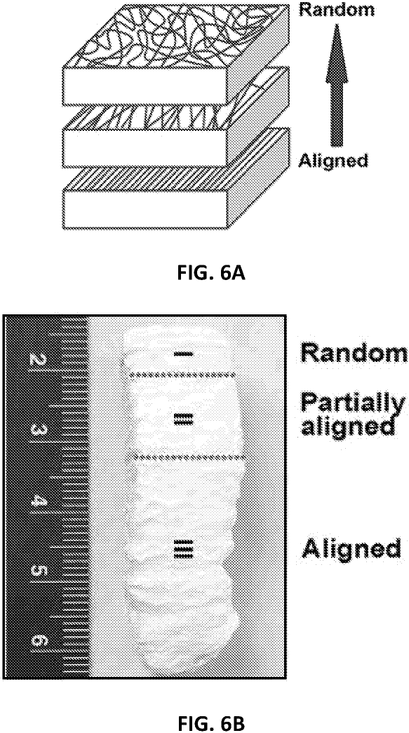

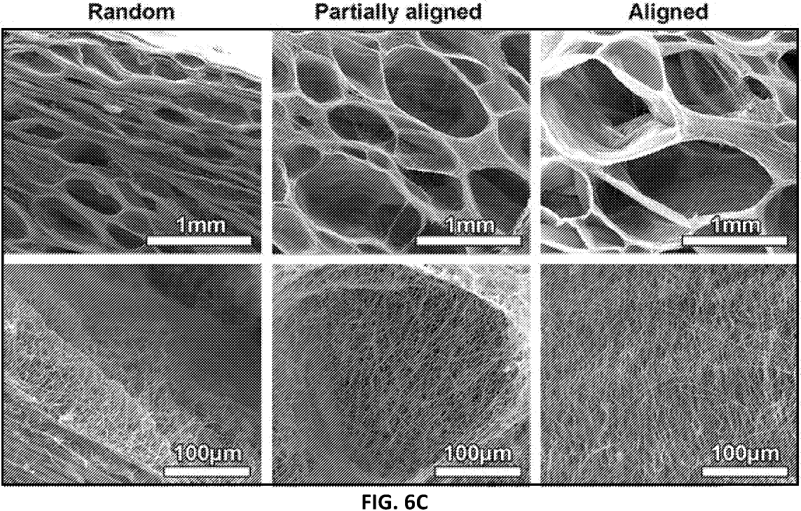

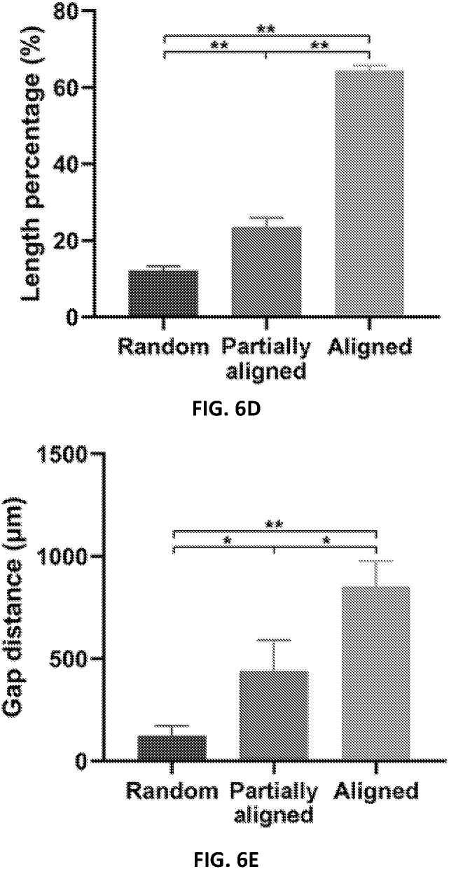

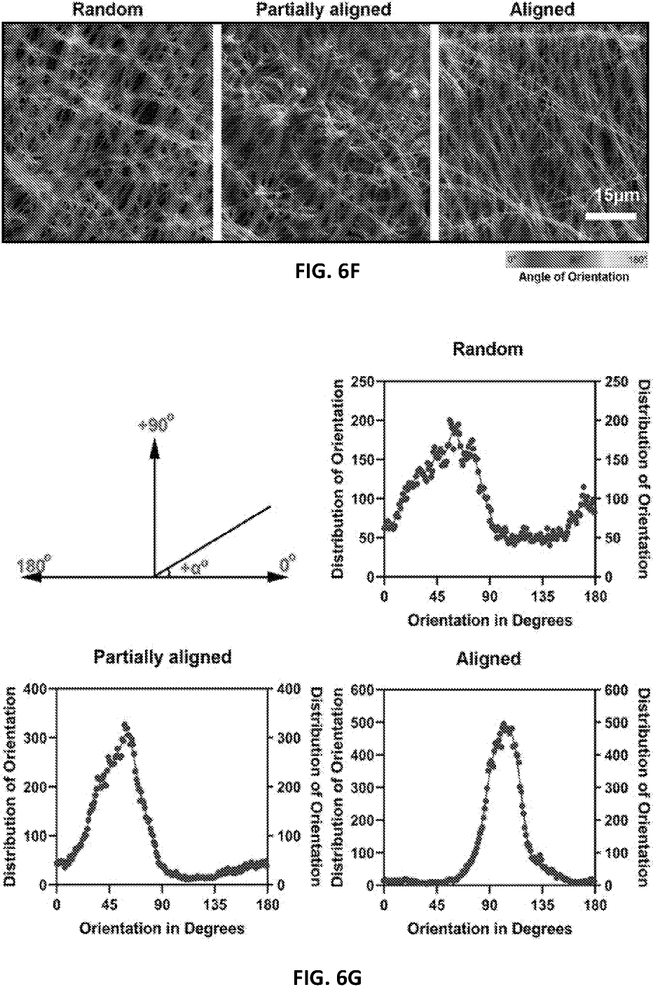

In addition to 3D nanofiber assemblies with gradients in pore sizes as above-mentioned, 3D expanded nanofiber scaffolds with dual gradations in both pore sizes and fiber organizations were also prepared. Briefly, a 1-mm thick PCL nanofiber mat was first produced by sequential deposition of random fibers, partially aligned fibers, and aligned fibers by increasing the rotating speed of the mandrel during electrospinning (FIG. 6A). Next, the fiber mat was expanded to form 3D nanofiber scaffolds using the gas-foaming technology (FIG. 6B) (Jiang, et al., ACS Biomater. Sci. Eng. (2015) 1:991-1001; Jiang, et al., Adv. Healthc. Mater. (2016) 5:2993-3003; Chen, et al., Biomaterials (2018) 179:46-59). FIG. 6C shows SEM images of the cross sections of expanded 3D scaffolds, revealing that pore size increased as fiber alignment increased from randomly oriented, to partially aligned, and to uniaxially aligned. The length percentages of each region were 13.11 ± 0.97%, 21.31 ± 2.58%, and 65.57 ± 1.29% (FIG. 6D). Corresponding maximum pore sizes were 98.04 ± 47.62, 460.78 ± 160.77, and 843.13 ± 130.89 µm (FIG. 6E). Fiber orientation in each region was computed via OrientationJ, an Image J plugin, which confirmed the gradation of fiber organizations from regions I to III (FIGS. 6F and 6G). Gradation in pore sizes is attributed to disparity of expansion rates between random, partially aligned, and aligned nanofiber regions, as aligned nanofiber membranes may require smaller forces to expand between nanofiber layers, while random nanofiber membranes require larger forces to separate the sequentially deposited layers due to the fiber entanglement. Scaffolds with dual gradients can be used to recapitulate the native ECM structure while regulating cell response. In addition, 3D scaffolds with graded fiber alignment in parallel can be generated through expansion in a customized mold, coating, and/or crosslinking (Chen, et al., Appl. Phys. Rev. (2020) 7:021406; Chen, et al., Nano Lett. (2019) 19:2059-2065; Jiang, et al., Acta Biomater. (2018) 68:237-248). These scaffolds can be used to closely mimic ECM fiber organizations at tissue interfaces such as tendon-to-bone insertion site (Rossetti, et al., Nat. Mater. (2017) 16:664-670).

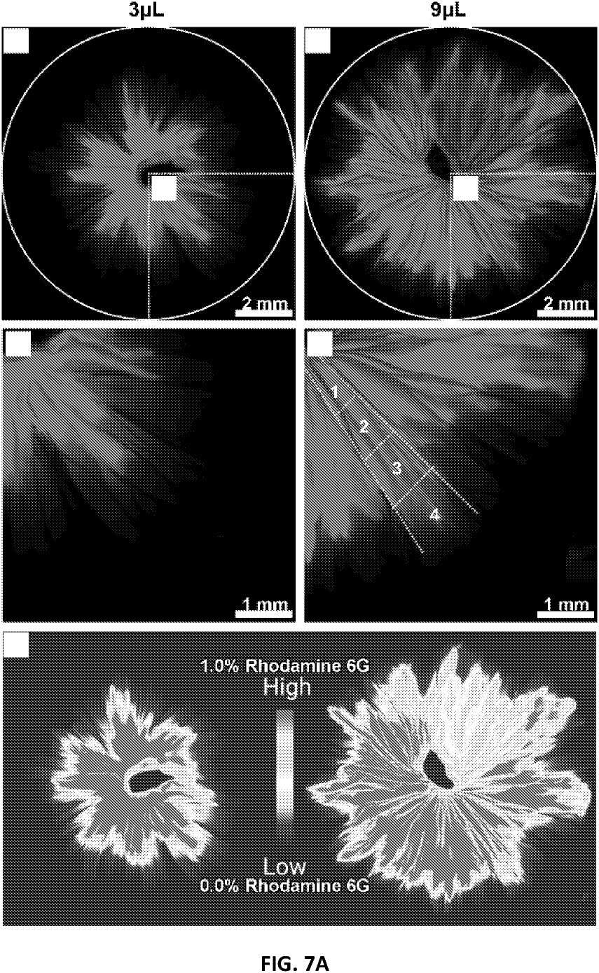

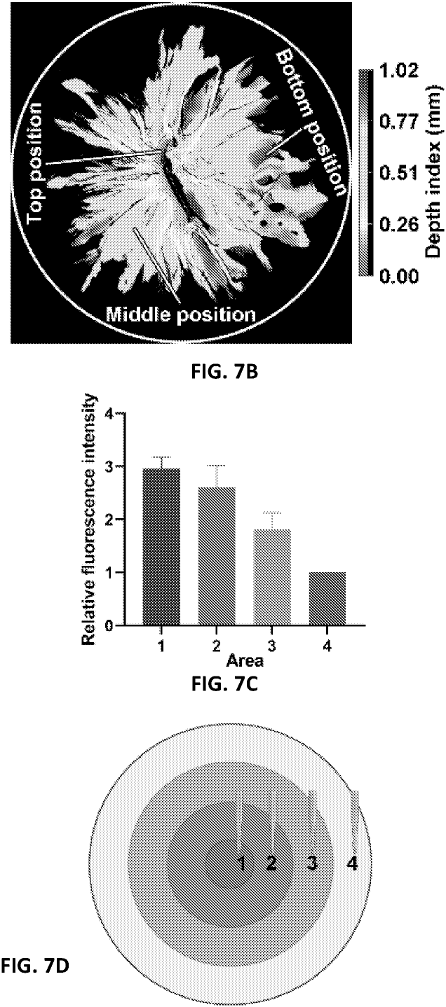

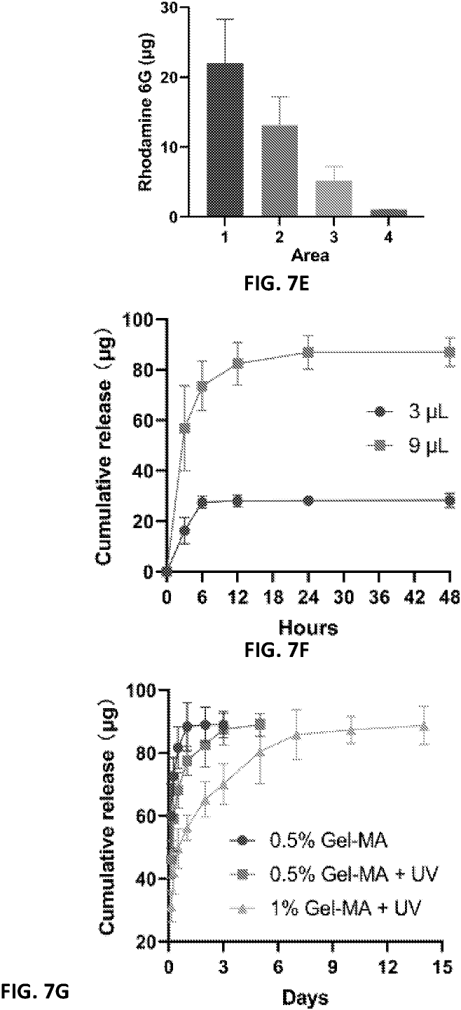

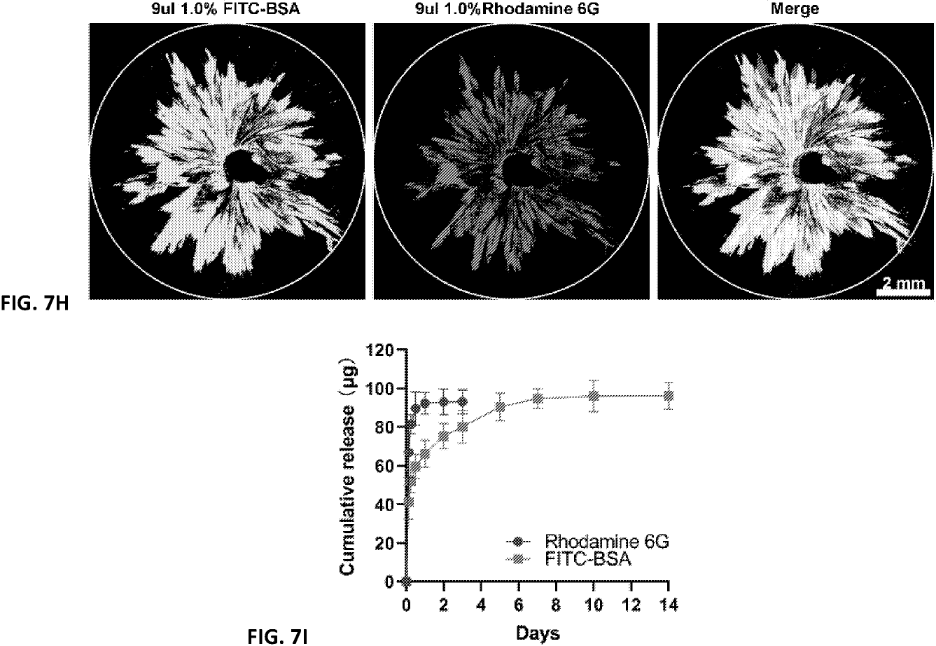

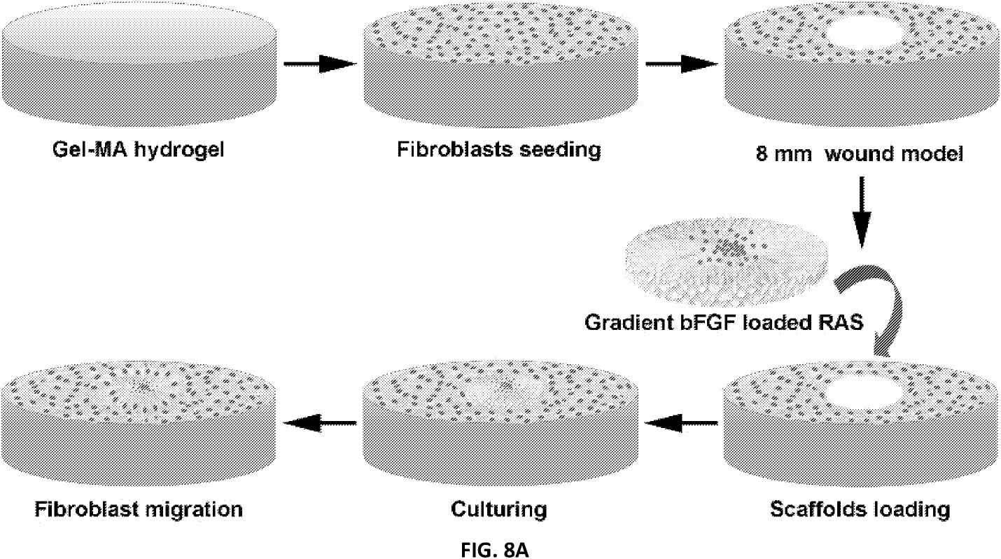

In addition to the structural gradient, 3D nanofiber assemblies with compositional gradients were fabricated. 2D electrospun membranes were transformed into 3D scaffolds consisting of radially aligned nanofibers by solids-of-revolution inspired expansion (Chen, et al., Nano Lett. (2019) 19:2059-2065). Radially-aligned pores were capable of guiding and promoting cell migration from the surrounding tissue to the center of the scaffolds, accelerating wound healing. To demonstrate the formation of compositional gradient, rhodamine aqueous solution was dripped to the center of the transformed 3D scaffold and allowed to diffuse outward. Fluorescent images showed a gradation in rhodamine 6G content along the radial direction of the 3D scaffolds (FIG. 7A). Rhodamine 6G diffusion area was increased by increasing the volume of dripped rhodamine solution from 3 µL to 9 µL (FIG. 7A). FIG. 7A also shows the false-color images of 3D scaffolds, allowing visualization of the rhodamine 6G gradients by fluorescent intensity heatmapping. In addition, the rhodamine 6G diffusion also occurred along the axial direction from top to bottom with a depth of 1.0 mm which was equal to the thickness of the scaffold (FIG. 7B). To quantify rhodamine 6G gradation, scaffolds were divided by four regions and the relative fluorescence intensities were measured, indicating a gradual decrease from the center to the edge (FIG. 7C). To further quantify the rhodamine 6G gradation, ten samples of each region were collected by punching (FIG. 7D). The total amount of rhodamine 6G in each region from the center to the edge was 23.12 ± 6.34 µg, 13.76 ± 4.21 µg, 5.02 ± 3.50 µg, and 1.17 ± 0.43 µg, respectively (FIG. 7E). Quantified rhodamine amounts showed a similar trend as fluorescent intensity analysis. The cumulative release profiles of rhodamine 6G from 3D gradient scaffolds showed an initial burst release, with a plateau arising within 6 and 24 h, respectively (FIG. 7F). Blending Gel-MA with the rhodamine 6G solution and crosslinking can be used to reduce the release rate of rhodamine 6G (FIG. 7G).

In addition to the gradient of single compound, multi-composition gradients can be achieved based on the same principle. FIG. 7H shows the fluorescent images of a 3D radially aligned nanofiber scaffold with dual gradations in rhodamine 6G (red) and FITC-BSA (green) content along the radial direction by successively dripping 1% FITC-BSA Gel-MA solution, crosslinking, and adding 0.3% rhodamine 6G solution. All the rhodamine 6G released within 1 day, while FITC-BSA showed a sustained release over 7 days (FIG. 7I).

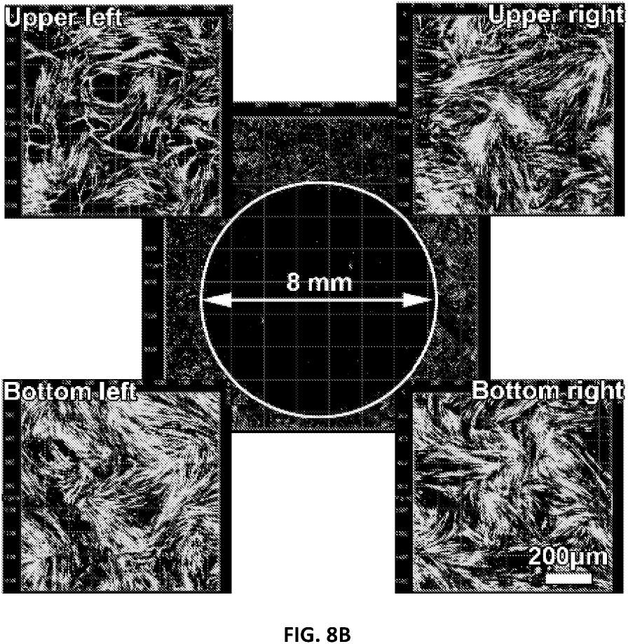

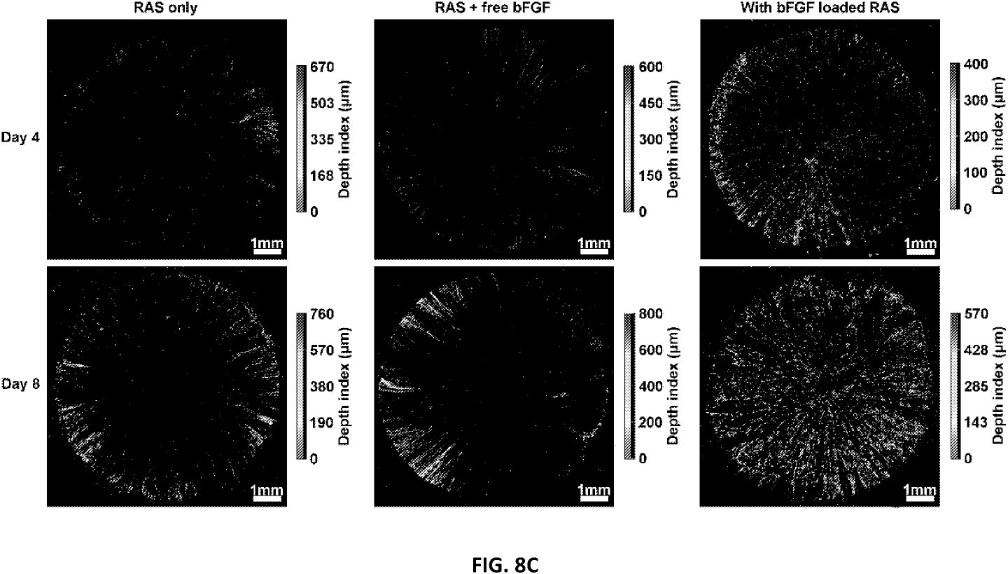

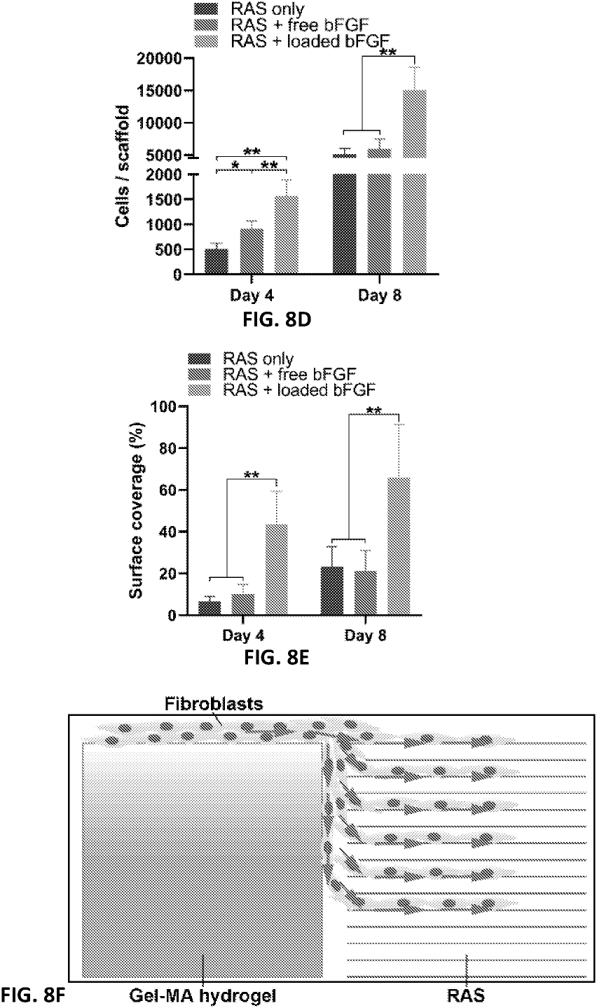

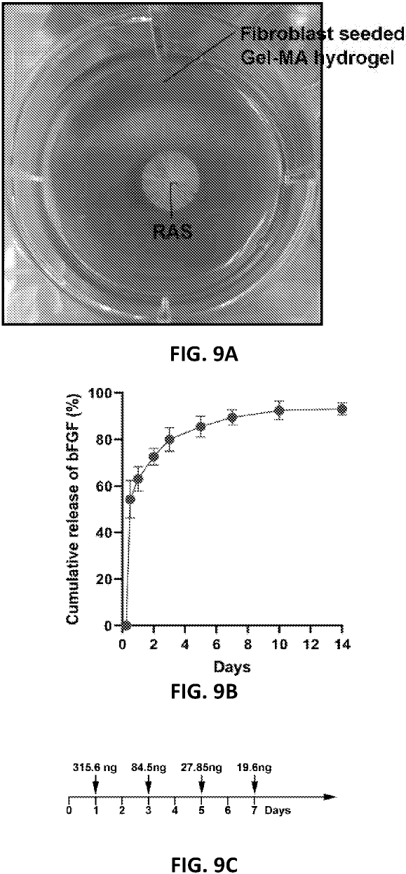

To examine the effect of compositionally-graded scaffolds on cell response, 3D radially aligned nanofiber scaffolds with graded basic fibroblast growth factor (bFGF) were created using the method above-mentioned as bFGF can promote cell migration, angiogenesis, and ECM deposition during wound healing (Yun, et al., J. Tissue Eng. (2010) 1:218142). To illustrate the recruitment of cells towards the center of the scaffold, an in vitro wound model (8 mm diameter) was first created by punching Gel-MA hydrogels and seeding with GFP-labeled human dermal fibroblasts. After seeded cells surrounding the defect reached 85% confluency, the 3D radially aligned nanofiber scaffold (8 mm) with graded bFGF was inserted into the defect (FIGS. 8A, 8B and 9). FIG. 8C shows fluorescent images indicating that more fibroblasts were detected on the bFGF gradient scaffold compared to scaffolds without and with supplement of equivalent dose of free bFGF after 4 and 8 days of incubation. Moreover, detected fibroblasts showed a spatially diverse distribution in all groups. The total number of detected fibroblasts (FIG. 8D) and cell-containing surface coverage (FIG. 8E) of bFGF gradient scaffolds were markedly higher than the scaffolds with and without supplement of free bFGF. Cells on the surface of Gel-MA hydrogels could not only migrate directly to the surface of 3D scaffolds, but also migrate downwards along the wall of the punched hole, simultaneously migrating into the scaffolds at varying depths, as evidenced by the 3D distribution of detected fibroblasts (FIG. 8F). Based on the same strategy, nanofiber assemblies with gradients in other growth factors, cytokines, peptides, DNA plasmids, microRNA, and siRNA can be used for enhancing their biological functions and facilitating wound healing and tissue regeneration. Furthermore, the combination of multiple biological molecules can achieve better therapeutic effects (Chen, et al., Biomaterials (2010) 31:6279-6308). 3D nanofiber assemblies with gradient in multiple compositions (e.g., two or more growth factors) can be produced based on this strategy, which will allow spatial control of various signaling molecules for effectively regulating cell response.

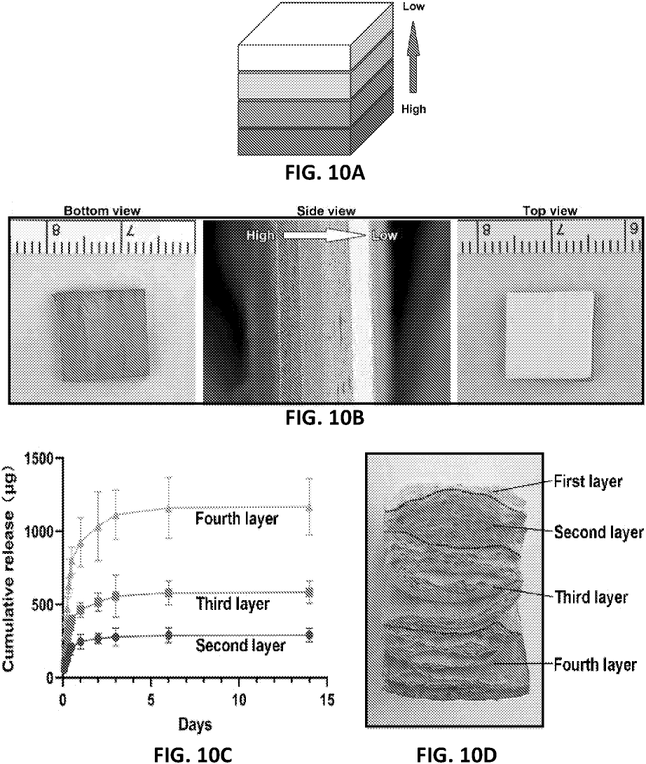

Another method to fabricate compositionally-graded scaffolds is by expanding nanofiber mats consisting of sequentially deposited nanofiber layers using rapid depressurization of subcritical CO₂ fluids (FIG. 10) (Jiang et al. (2018) Acta Biomater., 68:237-248). This strategy will retain the bioactivity and prevent the loss of encapsulated biological molecules, as well as achieve sustained release over a longer period of time than the aforementioned method.

In summary, new methods for fabricating 3D nanofiber assemblies with gradients in structure and composition have been provided. The 3D nanofiber scaffolds with gradients in pore sizes were created by controlling the expansion rate of different regions of 2D nanofiber mats by blending various amounts of Pluronic® F-127. The dual gradients in pore sizes and fiber organizations from random, to partially aligned, and to aligned were formed by expanding 2D mats consisting of nanofiber layers sequentially deposited onto the mandrel with gradually increasing rotating speeds. In addition, the compositional gradients on 3D nanofiber assemblies were prepared based on the diffusion and encapsulation. It was also demonstrated that dense regions of expanded scaffolds with graded pore sizes can greatly enhance the expression of hypoxia-related markers and chondrogenic differentiation of BMSCs, and bFGF gradients on 3D radially aligned nanofiber scaffolds can significantly accelerate the migration of human dermal fibroblasts. The 3D nanofiber assemblies with gradations in structure and composition show great ability in regulating cell responses with applications in wound healing, tissue repair and regeneration, and tissue modeling.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of synthesizing a nanofiber structure comprising regions with different pore sizes, said method comprising:
a) electrospinning a first nanofiber region with a polymer and a first amount of a surfactant and a second nanofiber region with a polymer and a second amount of a surfactant; and
b) expanding the electrospun nanofiber structure synthesized in step a) by gas foaming,
wherein the first amount of a surfactant is different than the second amount of a surfactant, thereby synthesizing said nanofiber structure comprising regions with different pore sizes.

2. The method of claim 1, wherein step a) comprises electrospinning further nanofiber regions with a polymer and a surfactant.

3. The method of claim 1, wherein said surfactant is an amphiphilic block copolymer.

4. The method of claim 3, wherein said surfactant is a poloxamer.

5. The method of claim 4, wherein said surfactant is poloxamer 407.

6. The method of claim 1, wherein said polymer is a hydrophobic polymer.

7. The method of claim 6, wherein said polymer is polycaprolactone.

8. The method of claim 1, wherein the first nanofiber region and the second nanofiber region are layers having the same length and width.

9. The method of claim 1, wherein the polymer and the surfactant are the same in all of the regions of nanofiber structure.

10. A method of synthesizing a nanofiber structure comprising regions with different nanofiber alignments, said method comprising:
a) electrospinning a first nanofiber region with a polymer with a first nanofiber alignment and a second nanofiber region with a polymer with a second nanofiber alignment; and
b) expanding the electrospun nanofiber structure synthesized in step a) by gas foaming,
wherein the first nanofiber alignment is different than the second nanofiber alignment, thereby synthesizing said nanofiber structure comprising regions with different nanofiber alignments.

11. The method of claim 10, wherein step a) comprises electrospinning further nanofiber regions with a polymer with a nanofiber alignment.

12. The method of claim 10, wherein said polymer is a hydrophobic polymer.

13. The method of claim 12, wherein said polymer is polycaprolactone.

14. The method of claim 10, wherein the first nanofiber region and the second nanofiber region are layers having the same length and width.

15. The method of claim 10, wherein the polymer is the same in all of the regions of nanofiber structure.

16. The method of claim 10, wherein the nanofiber alignments are selected from the group consisting of aligned nanofibers, partially aligned nanofibers, random nanofibers, and entangled nanofibers.

17. A method of synthesizing a nanofiber structure comprising a compositional gradient of at least one agent, said method comprising:
a) electrospinning a nanofiber mat or membrane comprising radially aligned nanofibers;
b) expanding the electrospun nanofiber structure synthesized in step a) by gas foaming; and
c) adding the at least one agent to the center of the expanded nanofiber structure, wherein said at least one agent radially diffuses through the expanded nanofiber structure to form said nanofiber structure comprising a compositional gradient of at least one agent.

18. The method of claim 17, wherein said nanofibers comprise a hydrophobic polymer.

19. The method of claim 18, wherein said hydrophobic polymer is polycaprolactone.

20. The method of claim 1, further comprising coating the nanofibers and/or the nanofiber structure with a hydrogel or gelatin.

21. The method of claim 1, further comprising crosslinking or thermally treating the nanofiber structure.

22. The method of claim 1, further comprising adding cells, tissues, and/or a bioactive agent to the nanofiber structure.

23. The method of claim 22, wherein said bioactive agent is selected from the group consisting of a therapeutic agent, a growth factor, a signaling molecule, a cytokine, a hemostatic agent, an antimicrobial, and an antibiotic.

24. The nanofiber structure synthesized by the method of claim 1.

25. A method for treating and/or preventing a disease or disorder in a subject in need thereof, said method comprising administering to said subject the nanofiber structure of claim 24.

26. The method of claim 25, wherein said disease or disorder is bleeding, optionally wherein said nanofiber structure comprises a blood clotting factor.

27. The method of claim 25, wherein said disease or disorder is bone or cartilage loss, optionally wherein the nanofiber structure comprises cells and/or bone stimulating agent.
